(19) United States
(12) Patent Application Publication
(10) Pub. No.: US 2023/0386672 A1
(43) Pub. Date: Nov. 30, 2023

(54) COMPUTER-BASED SYSTEMS CONFIGURED FOR CONDITION MEASUREMENT PREDICTION AND METHODS THEREOF

(71) Applicant: THE FEINSTEIN INSTITUTES FOR MEDICAL RESEARCH, INC., Manhasset, NY (US)

(72) Inventors: Theodoros ZANOS, Roslyn Heights, NY (US); Jamie HIRSCH, Great Neck, NY (US); Viktor TOTH, Flushing, NY (US)

(21) Appl. No.: 18/028,258

(22) PCT Filed: Sep. 22, 2021
(86) PCT No.: PCT/US2021/051567

§ 371 Date: Mar. 24, 2023

Related U.S. Application Data
(60) Provisional application No. 63/082,642, filed on Sep. 24, 2020.

(51) Int. Cl. G16H 50/30; A61B 5/00; A61B 5/0205

(52) U.S. Cl. CPC G16H 50/30; A61B 5/4806; A61B 5/02055

(57) ABSTRACT

Systems and methods of the present disclosure include processing devices to receive patient vital measurement data, including time-sequences of respiration rate measurements, heart rate measurements, blood pressure measurements, and temperature measurements, during a preceding time period preceding a prediction period. The processing devices utilize a risk prediction model to predict a probability of a low risk classification for the prediction period based on the vital measurements of the preceding time period. The processing devices produce a risk classification for the prediction period based on a probability of the low risk classification exceeding a classification probability threshold, where the risk classification includes one of a low-risk classification and a not-low-risk classification. The processing devices use the classification to generate a vital measurement schedule modification and display the vital measurement schedule modification to practitioners, including a wake-up time associated with each patient for further vital measurements.

### CROSS-REFERENCE TO RELATED APPLICATIONS

This application claims priority to U.S. Provisional Application 63/082,642 filed on Sep. 24, 2020, which is herein incorporated by reference in its entirety.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in drawings that form a part of this document: Copyright, Northwell Health, All Rights Reserved.

### FIELD OF TECHNOLOGY

The present disclosure generally relates to improved computer-based systems configured for one or more novel technological applications of prediction condition measurements, such as the severity of condition indicator measurements in healthcare patients, and methods of thereof.

### BACKGROUND OF TECHNOLOGY

Poor sleep is associated with a range of untoward effects that range from delirium and cognitive impairment, to weakened immunity, hypertension, elevated stress hormones and increased mortality. The underlying causes of impaired sleep are multifactorial and include environmental factors such as noise, light, and patient care activities. Overnight vital sign monitoring, specifically, has been identified as an important cause of fragmented sleep. The frequency of routine vital signs assessment is commonly every 4 to 5 hours for medical and surgical patients. However, a reduction in overnight vital sign measurements and medication administration demonstrated fewer sleep interruptions and improved patient experience for some patients.

While overnight vitals disrupt sleep, they are clearly needed for patients who benefit from close monitoring of vital signs in order to detect subtle signs of clinical deterioration. However, typically, identifying these high-risk patients that may need additional monitoring or otherwise may experience clinical deterioration include various techniques with various predictive ability. By contrast, identifying the low-risk cohort unlikely to benefit from such additional care remains unaddressed.

### SUMMARY OF DESCRIBED SUBJECT MATTER

In some embodiments, the present disclosure provides an exemplary technically improved computer-based method that includes at least the following steps of receiving, by at least one processor, patient vital measurement data for each patient of a plurality of patients, where the each patient vital measurement data includes a vital measurement time-sequence, including: i) respiration rate measurements, ii) heart rate measurements, iii) blood pressure measurements, and iv) temperature measurements; determining, by the at least one processor, a sub-score for each vital measurement of the vital measurement sequence for each patient; determining, by the at least one processor, a risk score for each vital measurement of the vital measurement sequence for each patient based on a sum of each sub-score for each vital measurement to produce a record of risk scores through time for each patient; receiving, by the at least one processor, a label for each predetermined time period of the vital measurement sequence based on a comparison of each risk score in each predetermined time period with a risk threshold, where the label includes a low risk label where one or more risk scores of each vital measurement is below the risk threshold; utilizing, by the at least one processor, a recurrent neural network to predict a probability of a low risk classification for each predetermined time period based at least in part on a subset of the plurality of vital measurements preceding each predetermined time period; producing, by the at least one processor, a risk classification for each predetermined time period based on a respective probability of the low risk classification exceeding a classification probability threshold, where the risk classification includes one of a low-risk classification and a not-low-risk classification; determining, by the at least one processor, classification error based on a comparison of a respective risk classification and a respective label for each predetermined time period, where an incorrect low-risk classification is weighted eight times more than an incorrect not-low-risk classification; and determining, by the at least one processor, updated weights of long short-term memory (LSTM) cell of the recurrent neural network based on the classification error to train the recurrent neural network.

In some embodiments, the present disclosure provides another exemplary technically improved computer-based method that includes at least the following steps of receiving, by at least one processor, patient vital measurement data for each patient of a plurality of patients, where the each patient vital measurement data includes a vital measurement time-sequence, including: i) respiration rate measurements, ii) heart rate measurements, iii) blood pressure measurements, and iv) temperature measurements; and where the vital measurement sequence includes a plurality of vital measurements during a preceding time period preceding a prediction period; utilizing, by the at least one processor, a recurrent neural network to predict a probability of a low risk classification for the prediction period based at least in part on the plurality of vital measurements of the preceding time period; producing, by the at least one processor, a risk classification for the prediction period based on a probability of the low risk classification exceeding a classification probability threshold, where the risk classification includes one of a low-risk classification and a not-low-risk classification; generating, by the at least one processor, a vital measurement schedule modification based on the risk classification; and causing to display, by the at least one processor, the vital measurement schedule modification to at least one practitioner, where the vital measurement schedule modification indicates a wake-up time associated with each patient for at least one next vital measurement.

In some embodiments, the present disclosure provides another exemplary technically improved computer-based product that includes at least the following components a non-transitory computer readable medium having software instructions stored thereon. Execution of the software instructions cause at least one processor to perform steps to: receive patient vital measurement data for each patient of a plurality of patients, where the each patient vital measurement data includes a vital measurement time-sequence, including: i) respiration rate measurements, ii) heart rate measurements, iii) blood pressure measurements, and iv) temperature measurements; and where the vital measurement sequence includes a plurality of vital measurements during a preceding time period preceding a prediction period; utilize a recurrent neural network to predict a probability of a low risk classification for the prediction period based at least in part on the plurality of vital measurements of the preceding time period; produce a risk classification for the prediction period based on a probability of the low risk classification exceeding a classification probability threshold, where the risk classification includes one of a low-risk classification and a not-low-risk classification; generate a vital measurement schedule modification based on the risk classification; and cause to display the vital measurement schedule modification to at least one practitioner, where the vital measurement schedule modification indicates a wake-up time associated with each patient for at least one next vital measurement.

In some embodiments, the present disclosure provides another exemplary technically improved computer-based system that includes at least the following components of at least one processor. The at least one processor is configured to perform steps to: receive patient vital measurement data for each patient of a plurality of patients, where the each patient vital measurement data includes a vital measurement time-sequence, including: i) respiration rate measurements, ii) heart rate measurements, iii) blood pressure measurements, and iv) temperature measurements; and where the vital measurement sequence includes a plurality of vital measurements during a preceding time period preceding a prediction period; utilize a recurrent neural network to predict a probability of a low risk classification for the prediction period based at least in part on the plurality of vital measurements of the preceding time period; produce a risk classification for the prediction period based on a probability of the low risk classification exceeding a classification probability threshold, where the risk classification includes one of a low-risk classification and a not-low-risk classification; generate a vital measurement schedule modification based on the risk classification; and cause to display the vital measurement schedule modification to at least one practitioner, where the vital measurement schedule modification indicates a wake-up time associated with each patient for at least one next vital measurement.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIGS. 1-20 show one or more schematic flow diagrams, certain computer-based architectures, and/or screenshots of various specialized graphical user interfaces which are illustrative of some exemplary aspects of at least some embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

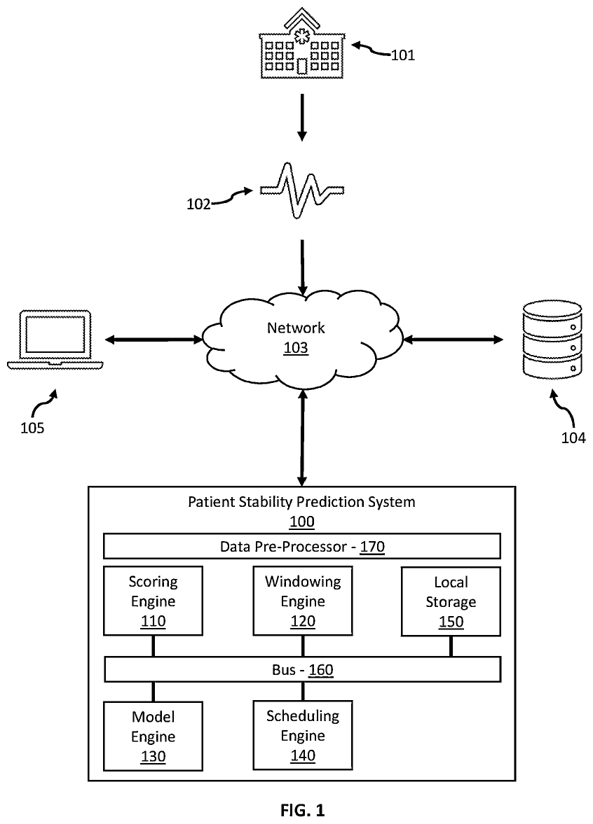

FIGS. 1 through 14 illustrate systems and methods of automatic patient care schedule generation using patient condition measurement prediction. The following embodiments provide technical solutions and technical improvements that overcome technical problems, drawbacks and/or deficiencies in the technical fields involving patient condition measurement and tracking tools, and patient care management systems and platforms. As explained in more detail, below, technical solutions and technical improvements herein include aspects of improved machine learning techniques for vital measurement, vital measurement tracking, and vital measurement prediction with greater accuracy and efficiency for the new capability to automatically identify low risk patients while improving scheduling tools for patient care scheduling based on automatically identified low risk patients according to the vital measurement prediction. Based on such technical features, further technical benefits become available to users and operators of these systems and methods. Moreover, various practical applications of the disclosed technology are also described, which provide further practical benefits to users and operators that are also new and useful improvements in the art.

FIG. 1 is a block diagram of an exemplary computer-based system for predicting condition measurements to automatically establish patient care schedules in accordance with one or more embodiments of the present disclosure.

In some embodiments, a healthcare facility 101 may be connected to a communication network 103 for interfacing with a patient stability prediction system 100. In some embodiments, the healthcare facility 101 produces patient vital measurement data 102 for each patient in the healthcare facility 101 to assess and monitor patient conditions. In some embodiments, the vital measurement data 102 can include vital measurements including, e.g., respiration rate, heart rate, blood pressure, temperature, electrocardiographic (ECG) data, electrodermal activity (EDA), electroencephalography (EEG) data, among other vital measurements and combinations thereof. For example, the vital measurement data 102 can include measurements for respiration rate, heart rate, and temperature. In another example, the vital measurement data 102 includes measurements for respiration rate, heart rate and blood pressure. In yet another example, the vital measurement data 102 includes measurements for respiration rate, heart rate, blood pressure and temperature, as well as other more constant health markers, such as, e.g., age, weight, body mass index, height, sex, etc.

The vital measurement data 102 can include a time-sequence of measurement values for each type of vital measurement (e.g., respiration rate, heart rate, blood pressure, temperature, ECG, EDA, EEG, etc.) in, e.g., a continuous stream or periodic values of measurements by measurement devices at the healthcare facility 101. For example, the healthcare facility 101 may continuously monitor heart rate via an ECG measurement device or other heart rate measurement device, thus producing a continuous signal of time-varying values indicative of heart rate. However, in some embodiments, the heart rate may be periodically measured, thus produced periodic time-varying values. For example, the healthcare facility 101 may produce a vital measurement data 102 value or set of values on a periodic basis including, e.g., once every hour, once every 2 hours, once every 4 hours, once every 5 hours, once every 6 hours, or other by some other period to produce a sequence of vital measurements through time for each patient to form the vital measurement data 102. In some embodiments, all vital measurements of the vital measurement data 102 for a given patient can be measured and produced on a uniform period. However, in some embodiments, some vital measurements may have different periods that other vital measurements. In some embodiments, the vital measurement data 102 is collected and produced on a same or a different period for each patient.

In some embodiments, the healthcare facility 101 provides the vital measurement data 102, via the network 103 to, e.g., a vital measurement database 104, a user computing device 105 associated with one or more healthcare providers, the patient stability prediction system 100, as well as other systems and devices, and any combination thereof. In some embodiments, the healthcare facility 101 may provide the vital measurement data 102 in real-time as the vital measurement data 102 is produced, or in batches at predetermined intervals of time, such as every night, every morning, every 12 hours, every 6 hours, every week, or by any other period. Thus, the healthcare facility 101 may provide the vital measurement data 102 as a sequence data, either as a continuous stream or in batches for time-varying and time-dependent vital measurement data 102.

In some embodiments, the vital measurement data 102 is provided to the network 103, e.g., in response to application programming interface (API) requests, according to a messaging protocol such as a publish-subscribe protocol or other messaging protocol. In some embodiments, the network 103 may include, e.g., a wired network of devices, such as, e.g., an ethernet network, universal serial bus (USB) connected devices, coaxial cable connections, fiber optic connections, among other wired connections to one or more devices, and combinations thereof. In some embodiments, the network 103 may include, e.g., a wireless network of devices, such as, e.g., one or more WiFi connected devices, one or more Bluetooth connected devices, one or more ZigBee connected devices, one or more Z-Wave connected devices, devices connected via a cellular network such as, e.g., Code-Division Multiple Access (CDMA), Global System for Mobile (GSM), Advanced Mobile Phone Systems (AMPS), 4G, 5G, or other cellular network and combinations thereof, among other wireless networking technologies and combinations thereof. In some embodiments, the network 103 may include, e.g., the Internet, an intranet, a local area network, a wireless local area network (WLAN), a wide area network (WAN), a wireless wide area network (WWAN), or other networking technique and combinations thereof.

In some embodiments, the vital measurement data 102 may be provided to the vital measurement database 104 for logging and recording vital measurements for each patient. Accordingly, a user, such as healthcare provider including, e.g., a doctor or nurse, may access a particular patient's vital measurements and a history thereof to assist in determining patient condition and condition deterioration or improvement.

In some embodiments, the user may access the vital measurement data 102 using the user computing device 105. In some embodiments, the user computing device 105 may be one of multiple user computing devices 105 in communication with the network 103. Each user computing device 105 may receive and display vital measurement data 102, e.g., in real-time from the healthcare facility 101, or by accessing the vital measurement data 102 stored in the database 104, or both.

In some embodiments, the patient stability prediction system 100 may similarly receive the vital measurement data 102, e.g., in real-time from the healthcare facility 101, or by accessing the vital measurement data 102 stored in the database 104, or both. In some embodiments, the patient stability prediction system 100 may temporarily or permanently store the vital measurement data 102 in a local storage 150, such as, e.g., a data buffer, a cache, a random-access memory (RAM), a hard drive, a solid-state drive, a local database, or other data storage device or system. In some embodiments, the local storage 150 may provide the vital measurement data 102 for processing and analysis by a scoring engine 110, a windowing engine 120, a model engine 130, a scheduling engine 140, among other computer engines, software programs and processing devices. In some embodiments, each of the local storage 150, the scoring engine 110, the windowing engine 120, the model engine 130, the scheduling engine 140, among other components, may be in communication via a bus 160.

In some embodiments, the patient stability prediction system 100 includes a data pre-processor 170 to receive and pre-process the vital measurement data 102 from the network 103, from the healthcare facility 101, from the database 104, or a combination thereof. In some embodiments, the data pre-processor 170 may, e.g., receive patient records for each patient in the healthcare facility 101, where each patient record include the vital measurement data 102 for the respective patient. For example, data may be provided by the healthcare facility 101 in a de-identified format, include, e.g., 771,334 inpatient hospital visits amounting to 26,201,030 records of vitals in on example of an implementation of an embodiment. Each record may include, e.g., heart rate (HR), respiratory rate (RR), blood pressure (BP), body temperature (Tmpr), or other measurements, and combinations thereof, in addition to the corresponding risk scores.

In some embodiments, the data pre-processor 170 may remove outliers, including, e.g., possibly mistyped vitals, measurement errors, or other outliers, as well as impute missing values, provided that an earlier measurement is available within a predetermined time window, such as, e.g., a 12-hour time window. In some embodiments, the pre-processor 170 may also derive features, such as the elapsed time and change in risk score since the last recording, hour of day and the variance of risk sub-scores. In some embodiments, the pre-processor 170 may scale vitals and age variables to the range between 0 and 1, while time related features may be one hot encoded.

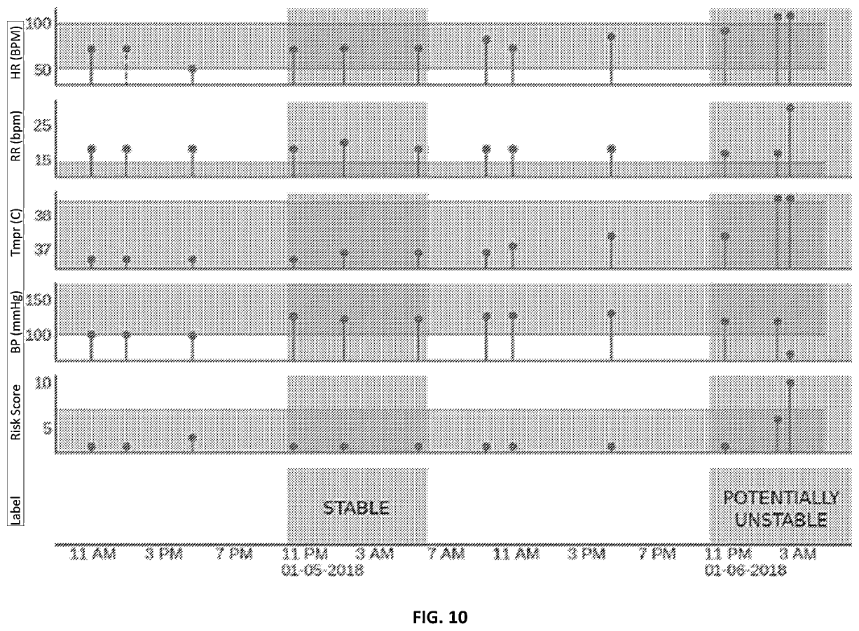

In some embodiments, each patient visit as recorded in the vital measurement data 102, may be represented as an array of vital measurement values (see, for example, FIG. 10). In some embodiments, the pre-processor 170 may extract sequences with a minimum of, e.g., 8 and the maximum length of, e.g., 42. Where vital measurements are taken every, e.g., 3-5 hours, this translates to a patient history of 32 hours for each patient, with the patient history of each patient being clipped at a maximum of, e.g., 7 days. In some embodiments, the data-preprocessor 170 may determine a binary variable for each overnight period, derived from the highest risk score of the 8-hour period following the corresponding patient history, e.g., if the score reached the threshold of 7, it was labelled positive, otherwise as negative.

In some embodiments, the vital measurement data 170 is split into training (70%), validation (15%) and test (15%) sets by patient visit, so the sequences drawn from one visit are only included in one of the data sets. While a prediction period for predicting patient stability may be restricted to the night hours in the validation and test sets, this restriction may be relaxed in the training set with the purpose of data augmentation.

In some embodiments, the scoring engine 110 may include a computer engine for determining condition scores associated with each patient based on each patient's vital measurement data 102 when such scores are not already present. In some embodiments, the computer engine can include one or more software or hardware components, or any combination thereof. In some embodiments, the hardware components can include, e.g., memory devices (e.g., cache, buffer, RAM, hard drives, solid state drives, or others) and processing devices (e.g., microprocessors, central processing units, graphic processing units, fully programmable gate arrays, neural processing units, tensor processing units, among others). In some embodiments, the scoring engine 110 includes a scoring engine-specific hardware components, or uses hardware components of the patient stability prediction system 100, or any combination thereof.

In some embodiments the scoring engine 110 includes software components, hardware components, or both that are arranged and configured to receive the vital measurement data 102, e.g., from the local storage 150 or directly from the network 103, and associated the vital measurement data 102 to one or more patients of the healthcare facility 101. For example, the vital measurement data 102 may include, e.g., metadata or data fields including a patient identifier of the patient associated with each measurement in the vital measurement data 102. Thus, at any given time, the vital measurement data 102 may include a set of vital measurements associated with a particular patient, the set of vital measurements including identifiers, e.g., in metadata or appended data fields, identifying the particular patient and the vital measured by each vital measurement in the set, as well as, e.g., a time stamp for each vital measured. In some embodiments, for any given time stamp, the vital measurement data 102 of a particular patient may include values indicating a measurement of vitals including, e.g., heart rate, respiration rate, blood pressure, temperature, electrodermal activity, or other biological and physiological measurements and combinations thereof, measured at that given time stamp.

In some embodiments, the set of vital measurements for a given patient at a given time stamp is indicative of the patient medical condition at that time stamp. Thus, in some embodiments, the scoring engine 110 may include software, hardware or combinations thereof specifically arranged and configured to determine from the set of vital measurements for each patient at each time stamp a score indicative of the patient condition or a risk of deterioration of condition.

In some embodiments, each type of vital measurement in a set of vital measurements may contribute to the score based on an impact of each type of vital measurement on the determination of patient condition deterioration risk. In some embodiments, the type of vital measurement may refer to the biometric or physiological measurement being measured, such as a type including heart rate measurements, a type including respiration rate measurements, a type including blood pressure measurement, a type including temperature measurements, among others. Thus, for example, a heart rate measurement and a respiration rate measurement may each be individually analyzed for an assessment of risk of deterioration.

Thus, in some embodiments, the scoring engine 110 may produce the score using, e.g., a sub-score determined for each type of vital measurement in the set of vital measurements. An example of a possible scoring method for sub-scores can include, e.g., the modified early warning score (MEWS), which forms a sub-score for each vital measurement, such as, e.g., systolic blood pressure, heart rate, respiratory rate, temperature, or “Alert, Voice, Pain, Unresponsive” responsiveness (AVPU) score, or combination thereof, based on predetermined ranges of each measurement. However, embodiments of the present invention may employ any scoring methodology, including MEWS, variations of MEWS or other scoring methods. In some embodiments, the sub-scores may then be aggregated for a given time stamp for each patient to determine the overall score reflecting the condition of each patient or a risk of deterioration of condition of each patient, or both, at that time stamp. In some embodiments, the sub-scores may be aggregated by, e.g., summing, averaging, determining the median or product of the sub-scores, or other aggregation method to determine a score. In some embodiments, the sub-scores may be weighted as part of the aggregation such that the effect of each sub-score on the final score reflects the strength of each vital measurement in assessing patient condition. In some embodiments, the weighting and/or aggregation may include, e.g., a learned model such as machine learning techniques including, e.g., a convolutional neural network, a recurrent neural network, a support vector machine, decision trees, gradient boosted trees, random forest, or other machine learning techniques. Accordingly, in some embodiments, the scoring engine 110 may determine for each patient at each time stamp of vital measurement data 102 a score for each patient indicative of a risk of deterioration of patient condition. In some embodiments, a higher score may represent a greater risk, indicative of a greater likelihood that a patient will experience imminent deterioration in health condition, thus indicating a greater need for care and monitoring.

In some embodiments, a patient may be deemed to have a risk of deterioration during a future time period, e.g., overnight, when their score throughout the night becomes equal to or exceeds 7, as calculated from vitals taken during the overnight period, e.g., between 10:00 PM and 6:00 AM. In some embodiments, however, the threshold may vary depending on the scoring methodology. Accordingly, a threshold may be selected that indicates the risk as more likely than not to have deterioration. In some embodiments, regardless of the risk scoring methodology, if the accumulated risk score (e.g., the taking all sub-scores into account), exceeds the threshold, the patient is deemed to have a risk of deterioration in the future time period. As shown in the example of FIG. 10, vital measurements are plotted against the time of the day they were taken. Based on the formula of the risk score calculation, certain ranges of values for the different vitals can be deemed as normal (not adding any values to the risk score).

In some embodiments, the windowing engine 120 may include a computer engine for producing batches of vital measurement data 102 or scores or both associated with each patient for use in predicting patient stability during a future prediction period. In some embodiments, the computer engine can include one or more software or hardware components, or any combination thereof. In some embodiments, the hardware components can include, e.g., memory devices (e.g., cache, buffer, RAM, hard drives, solid state drives, or others) and processing devices (e.g., microprocessors, central processing units, graphic processing units, fully programmable gate arrays, neural processing units, tensor processing units, among others). In some embodiments, the windowing engine 120 includes a windowing engine-specific hardware components, or uses hardware components of the patient stability prediction system 100, or any combination thereof.

In some embodiments, the patient stability prediction system 100 predicts patient stability for a given time period. To do so, the patient stability prediction system 100 employs vital measurement data 102 from a preceding period leading up to the period for which stability is predicted. For example, where the patient stability prediction system 100 produces patient stability predictions for a coming night or other future period, the patient stability predictions may be based on vital measurement data 102 and/or scores from a preceding period, including, e.g., the preceding set of vital measurement data 102, the previous 4 hours, the previous 6 hours, the previous 8 hours, the previous 10 hours, the previous 12 hours, the previous 24 hours, or other suitable period immediately preceding a current time. Thus, the windowing engine 120 may include the computer engine specifically arranged and configured to determine the vital measurement data 102 or scores or both associated with the preceding period for use in predicting the patient stability in the prediction period. For example, in some embodiments, the windowing engine 120 may extract all vital measurement data 102 and associated scores having time stamps in the preceding period, e.g., from the local storage 150 via the bus 160.

In some embodiments, the windowing engine 120 may employ a database query, e.g., using a database query language such as SQL or other language, or using a heuristic search, text string search, API call, or other data query technique to identify and retrieve all vital measurement data 102 and associated scores that have a time stamp in the preceding period.

In some embodiments, the windowing engine 120 provides time windows the vital measurement data 102, the scores, or both, to create, e.g., an input for the model engine 130 to predict patient stability for a future prediction period, or to create a training dataset for training the model engine 130, or both.

In some embodiments, to create a training dataset, the windowing engine 120 may access the vital measurement data 102, e.g., in the local storage 150, in the database 104, or both, and segment the vital measurement data 102 into preceding periods and prediction periods such that each prediction period has an associated preceding period that immediately precedes in time the respective prediction period. Thus, in some embodiments, the windowing engine 120 may identify using the time stamps, the vital measurement data 102 associated with each prediction period, such as, e.g., each night, e.g., between the hours of about 10:00 PM and about 6:00 AM, or between about 11:00 PM and about 5:00 AM, or other overnight time period during which patients may be sleeping. Each prediction period may then be paired with a window of vital measurement data 102 the precedes each overnight period or other sleeping hours period for a span of about, e.g., 8 hours or 12 hours, or other suitable preceding period. Thus, the windowing engine 120 may provide the window of vital measurement data 102 in the preceding period to, e.g., the model engine 130 to predict patient stability or condition for the following prediction period of each preceding period. The predicted patient stability or condition may then be compared against the window of vital measurement data 102 and associated scores in each corresponding prediction period to training the model engine 130 against the error of the prediction relative to the window of data.

In some embodiments, the windowing engine 120 may also provide windows of vital measurement data 102 and associated scores to the model engine 130 at a predetermined time ahead of a future prediction period such that the model engine 130 may generate patient stability or condition predictions for the coming prediction period. As such, at each predetermined time, such as, e.g., 1 hour, 2 hours, 3 hours, or other amount of time ahead of a next prediction period, such as, e.g., at before the start of a night shift, the windowing engine 120 may retrieve the vital measurement data 102 and associated scores for a preceding period of about, e.g., 8 hours or 12 hours, or other amount of time preceding the next prediction period.

In some embodiments, the model engine 130 uses a sequence of prior vitals for a given patient and predicts the chance of their deterioration for any given night, in order to determine the necessity of assessing overnight vitals. The algorithm ingests vital measurements (see, for example, FIG. 10), such as breathing rate (BR), heart rate (HR), blood pressure (BP) and temperature, as well as the patient's age and a Modified Early Warning (MEWS) risk score, such as the score generated by the scoring engine 110 as described above. A higher risk score, up to 15 for example, or up to 10, 20, 25, 30, 50, 100 or other scale, indicates instability in the patient's medical state and hence compels a more strenuous care involving frequent vital recordings, while lower scores closer to 0 indicate greater patient stability. In some embodiments, the model engine 130 is trained to predict the likelihood that a patient will experience a higher risk score, e.g., above a 7 on a scale of 0 to 15, or other threshold based on the scale, during the night, thus indicating decreased condition stability requiring greater monitoring by healthcare personnel. Or, in some embodiments, where the model engine 130 does not predict that the patient will experience a higher risk score, decreased or no monitoring may be required. In some embodiments, the model engine 130 may instead be trained that a patient will experience a low risk score, e.g., below a 7 on the scale, or other threshold, during the night, thus indicating a likelihood of remaining stable throughout the night.

In some embodiments, the model engine 130 may include a computer engine for prediction condition scores associated with each patient for the coming prediction based on each patient's vital measurement data 102 of the preceding period according to learned parameters. In some embodiments, the computer engine can include one or more software or hardware components, or any combination thereof. In some embodiments, the hardware components can include, e.g., memory devices (e.g., cache, buffer, RAM, hard drives, solid state drives, or others) and processing devices (e.g., microprocessors, central processing units, graphic processing units, fully programmable gate arrays, neural processing units, tensor processing units, among others). In some embodiments, the model engine 130 includes a model engine-specific hardware components, or uses hardware components of the patient stability prediction system 100, or any combination thereof.

In some embodiments, the model engine 130 may be configured to utilize one or more exemplary AI or machine learning techniques chosen from, but not limited to, recurrent or non-recurrent deep learning models, simple linear models (e.g., logistic regression), decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network or other suitable network. In some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model iteratively,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values, functions and aggregation functions. For example, an activation function of a node may be a step function, sine function, continuous or piecewise linear function, sigmoid function, hyperbolic tangent function, or other type of mathematical function that introduces nonlinear terms to the neural network. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, the model engine 130 may include a linear recurrent network. However, due to the nonlinearity of the vital measurement data 102 and risk scores, the model engine 130 may employ non-linear neural networks, such as non-linear recurrent neural networks incorporating, e.g., long short-term memory (LSTM), gated recovery units (GRU), or other recurrent neural network configurations.

In some embodiments, the scheduling engine 140 may include a computer engine for employing the model engine 130 predictions for each patient to generate automatic schedule adjustments for patient monitoring schedules. In some embodiments, the computer engine can include one or more software or hardware components, or any combination thereof. In some embodiments, the hardware components can include, e.g., memory devices (e.g., cache, buffer, RAM, hard drives, solid state drives, or others) and processing devices (e.g., microprocessors, central processing units, graphic processing units, fully programmable gate arrays, neural processing units, tensor processing units, among others). In some embodiments, the scheduling engine 140 includes a scheduling engine-specific hardware components, or uses hardware components of the patient stability prediction system 100, or any combination thereof.

In some embodiments, the scheduling engine 140 receives, e.g., via the bus 160, the prediction period predictions from the model engine 130 for each patient in the healthcare facility 101. Where the model engine 130 predicts that a patient will experience high instability (e.g., a score above 7 or other suitable score indicating high risk), the scheduling engine 140 may automatically select a high risk schedule for the patient. In some embodiments, a high risk schedule may include patient monitoring sessions, e.g., every hour, every two hours, every three hours, or every four hours. However, where the model engine 130 predicts a low risk of instability or condition deterioration, the scheduling engine 140 may automatically select a low risk schedule for the patient. For example, a low risk schedule may include, e.g., only one monitoring session overnight, thus facilitating improved sleep for the low risk patient, thus improving patient outcomes. Thus, the model engine 130 may provide stability predictions, e.g., each night before the start of a night shift. The predictions may incorporated into the clinical workflow by being integrated into tools that are already used by the nurses. In its simplest real-time productionalized form, a spreadsheet is sent to providers every evening indicating which patients are deemed able to sleep through the night. Other implementations of the algorithm may feature mobile apps with user-friendly screens that will enable nurses to visualize important patient information and retrieve the algorithm suggestions at their request.

In some embodiments, the scheduling engine 140 may automatically apply the selected patient monitoring schedule for each patient to an electronic rounds schedule of one or more healthcare personnel assigned to each patient, such as an electronic calendar. Accordingly, the scheduling engine 140 may automatically program an electronic schedule of each healthcare personnel with the rounds schedules. In some embodiments, the scheduling engine 140 may additionally or alternatively instruct a printer to print the selected patient monitoring schedule for each patient, or store the patient monitoring schedules for each patient for access by the user computing devices 105, or both.

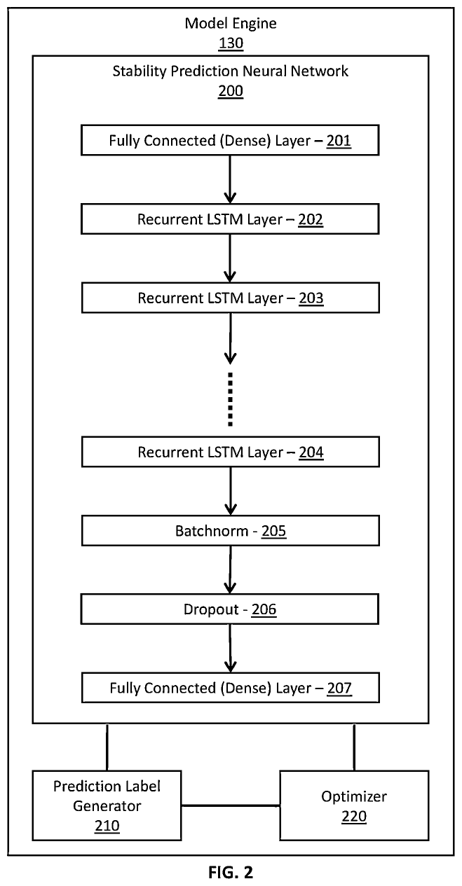

FIG. 2 is a block diagram of another exemplary computer-based system including a stability prediction neural network implemented by a model engine for predicting condition measurements to automatically establish patient care schedules in accordance with one or more embodiments of the present disclosure.

In some embodiments, the model engine 130 can include a stability prediction neural network 200 having an architecture configured to predict an overnight patient stability classification from a sequence of vital measurement data 102 and associated risk scores for each patient in a preceding period.

In some embodiments, the stability prediction neural network 200 includes a deep learning model to take the bundle of features sequentially and performs the prediction at the end of the sequence, where the sequence includes the time dependent values of the vital measurement data 102 and associated risk scores. Accordingly, to leverage the time-dependent nature of the sequences, the stability prediction neural network 200 may include of fully connected, or dense layer 201 at input, at least one recurrent long short-term (LSTM) memory layer 202 through 204, and a fully connected, or dense, layer 207 at output. In addition to fully connected layer 207 at output, the stability prediction neural network 200 may also include, e.g., a batch normalization layer 205 and a dropout layer 206.

In some embodiments, each layer of the stability prediction neural network 200 may having, e.g., a size of 256. While the stability prediction neural network 200 could include simple recurrent neural networks and bidirectional architectures, the stability prediction neural network 200 of FIG. 2 improves performance as well as improving training efficiency by orders of magnitude relative to the alternatives.

In some embodiments, incorporating the dropout layer 206 and the batch normalization layer 205 stabilizes model performance across instances within an ensemble of models, resulting in more robust generalization to test sets. In some embodiments, the stability prediction neural network 200 may be trained to predict the actual risk score in a probabilistic programming setting, which provides a certainty estimate aside from the risk estimate; however, such an embodiment provides increased complexity without an increase in benefit in the use-case of patient deterioration or stability prediction.

In some embodiments, the model engine 130 may include an ensemble of stability prediction neural networks 200, each with randomly initialized weights and shuffled training data. The combined prediction of the ensemble acts as a probability measure on whether the patient will be potentially unstable during the following night. Class imbalance may be handled by first removing samples from the negative, majority class until it constitutes a smaller subset of the training set, such as, e.g., 50%, 60%, 70% or other proportion based on the imbalance of the. Moreover, the error in predicting positive outcomes may be weighted higher in the loss function compared to the negative ones, or vice versa depending on the desired imbalance.

In some embodiments, the stability prediction neural network 200 generates a probability prediction for a patient becoming unstable. In some embodiments, a prediction label generator 210 may generate a stability classification based on the probability prediction. For example, in some embodiments, the prediction label generator 210 may label a patient as high risk where the probability exceeds a threshold of, e.g., 0.5, 0.6, 0.7 or other suitable threshold between 0 and 1. The label can be appended to a patient record or the vital measurement data 102 of the associated patient for use in generating the schedule for that patient in, e.g., a forthcoming overnight period.

In some embodiments, the model engine 130 may also include an optimizer 220 for training and evaluation of the stability prediction neural network 200. In some embodiments, the optimizer 220 may include an optimization function with backpropagation for training weights of the layers of the stability prediction neural network 200 based on error between the predictions and the actual patient stability throughout the night. In some embodiments, the error is determined using, e.g., a suitable objective function such as, e.g., cross-entropy loss, or other loss or error function. In some embodiments, cross-entropy facilitates implicitly forcing the stability prediction neural network 200 to output a probability measure that can be used to threshold the confidence of the output, but enabling fine tuning the false positives using the above described threshold. In some embodiments, in order to define the threshold in a clinically relevant manner, the threshold may be anchored to the number of false positive nights (let sleep, but potentially unstable) encountered out of the total of 10,000 nights (either positive or negative). Thus, the vital measurement data 102 of a training dataset may be delineated by the clinically applicable region to contain confidence thresholds resulting in less than 2 out of 10,000 missed nights. The threshold used in the prospective testing may be derived from the performance of the model on the retrospective test set, e.g., the threshold may be fixed on the edge of the clinically applicable region.

In some embodiments, the model engine 130 backpropagates the error using a suitable backpropagation algorithm, such as, stochastic gradient descent, an Adam optimizer, AdaGrad, RMSProp, among others. In some embodiments, the stability prediction neural network 200 may be re-trained on, e.g., a nightly, a weekly, or a monthly basis, or according to any other re-training schedule.

In some embodiments, using the predictions may enable salvaging approximately half of the patient nights, avoiding unnecessary vital assessment and leaving patients undisturbed overnight. These gains are achieved without significant risks, since the stability prediction neural network 200 misclassifies as stable only, e.g., 2 out of 10,000 patient nights. Moreover, by real time monitoring the HR of sleeping patients, the instances of potentially unstable sleeping patients can be further significantly reduced. Accordingly, the safety and accuracy of a machine learning based solution enables avoiding unnecessary overnight vital monitoring, resulting in reduced healthcare resource demand and improved efficiency of healthcare personnel deployment. The limited number of inputs needed also demonstrates the feasibility of deploying such an approach to the clinic, saving millions of sleepless patient nights, potentially improving patient outcomes and satisfaction, and further boosting health provider efficacy by reducing fatigue, enabling significant cost savings for health systems. Due to the large-scale dataset, the stability prediction neural network 200 is robust to different health care sites, patient demographics and conditions. Moreover, nurses or doctors may adjust the confidence threshold of the model to implement a more stringent or risk averse solution.

The benefits of reducing overnight vital sign monitoring extend beyond patient sleep. As healthcare systems seek to maximize efficiency and reduce waste, lean staffing models often hamper compliance with monitoring protocols as clinician capacity is exceeded. Adherence to vital sign protocols is clearly sensitive to staffing levels, with delayed or incomplete care occurring more frequently when staffing levels are low. Particularly at night, low nurse to patient ratios have been closely linked with low protocol compliance when compared with other times of day, when ratios are higher. Derivation of a tool, such as the present stability prediction neural network 200, to identify low-risk patients provides more consistent and standardized identification, and safely permit nurses and nurses' assistants to focus on the more acutely ill. It may also more closely align capacity and demand, which may positively impact provider stress and reduce burnout, while preserving or improving operational efficiency.

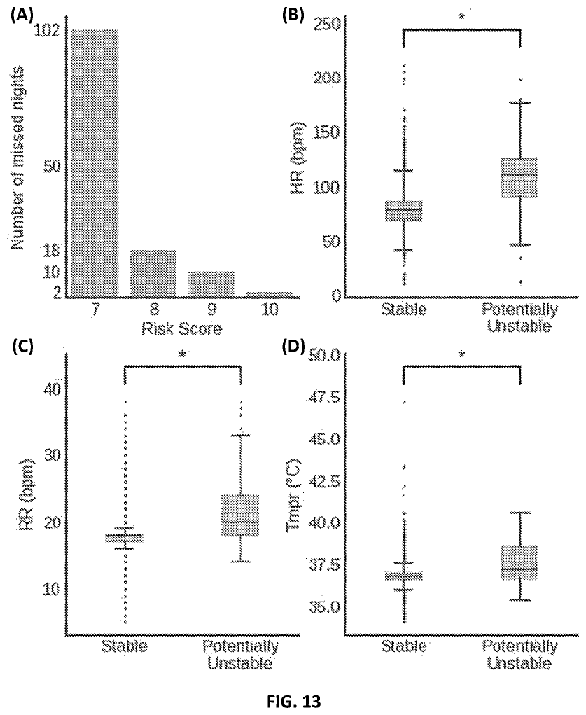

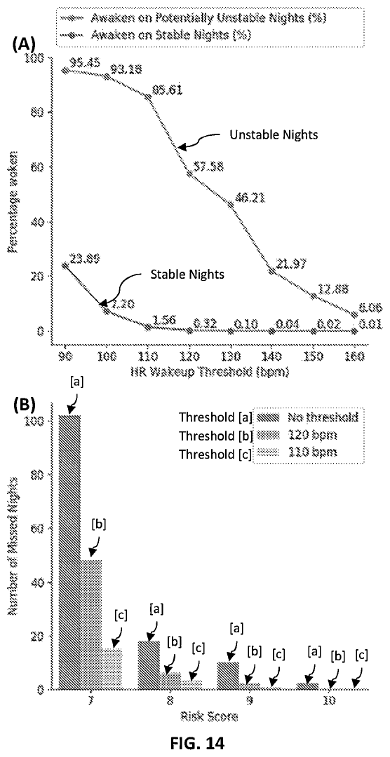

While the number of misclassified as stable patient nights of the stability prediction neural network 200 may be kept at a very low, clinically acceptable rate (e.g., about 2 in 10,000), this limitation could potentially deter some risk averse health providers from using it. As shown in FIG. 13, the overnight physiological state of a stable sleeping patient and of a potentially unstable misclassified patient is significantly different: RR, HR, and body temperature measures are considerably higher for the latter. Hence, a simple visual inspection of the sleeping patients should suffice in detecting some of the potentially misclassified. Based on the significant vital sign differences between stable and potentially unstable sleeping patients, real time HR monitoring will be able to identify unstable patient nights to further reduce the amount of misclassified nights (see, for example, FIG. 14). Moreover, the stability prediction neural network 200 relies on already existing data pipelines, and eliminates unnecessary vital measurements overnight. It does not require any additional data to be collected by providers, other than the vital values already collected to calculate risk scores.

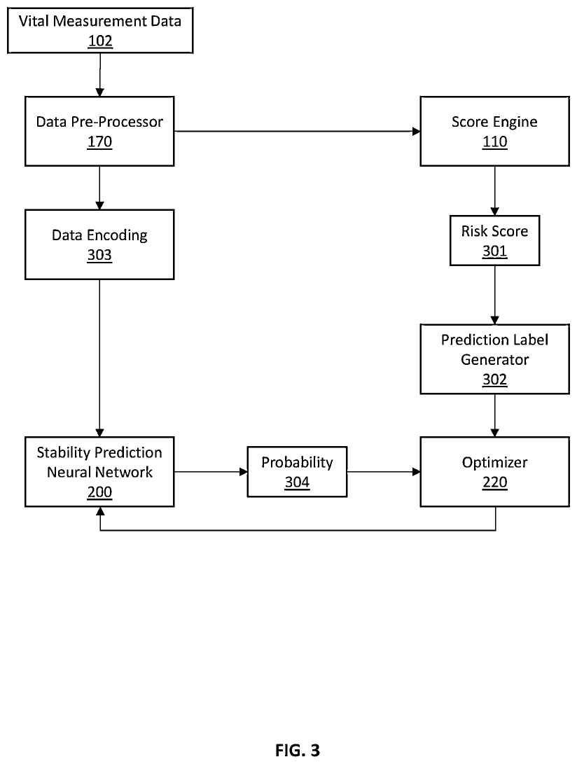

FIG. 3 illustrates a flowchart of an illustrative methodology for training a stability prediction neural network to predict condition measurements to automatically establish patient care schedules in accordance with one or more embodiments of the present disclosure.

In some embodiments, the data pre-processor 170 receives sequences of the vital measurement data 102 for each patient of the healthcare facility 101. In some embodiments, the vital measurement data 102 include sequences of, e.g., respiration rate measurements, heart rate measurements, blood pressure measurements, temperature measurements, among others. The data pre-processor 170 may cleanse and normalize the sequences of the vital measurement data 102, such as, e.g., removing outliers, imputing missing measurement (e.g., from a temporally near measurements, such as within about 12 hours of the missing measurement), among other pre-processing tasks.

In some embodiments, a dataset for training and testing the stability prediction neural network 200 may be constructed using the sequences of vital measurement data 102 for each patient. To do so, a ground truth dataset is built, whereby vital measurement data 102 for prediction periods is provided to the score engine 110. As described above, the vital measurement data 102 of the prediction periods may be extracted by the windowing engine 120 according to vital measurement time stamps being within an overnight period, such as between the hours of about 10:00 PM and 6:00 AM. In some embodiments, the score engine 110 may generate risk scores 301 at each time stamp of measurements in the sequence during the prediction periods. To produce the risk scores 301, the score engine 110 may calculate a sub-score for each vital measurement at the time stamp for each patient. For example, where the vital measurement data 102 includes respiration rate measurements, heart rate measurements, blood pressure measurements, and temperature measurements at each time stamp, the score engine 110 may calculate a risk sub-score for respiration rate at a particular time stamp, a risk sub-score for heart rate at the particular time stamp, and a risk sub-score for temperature measurement at the particular time stamp. In some embodiments, each sub-score may be indicative of a patient condition or risk of deterioration at the particular time stamp.

In some embodiments, the score engine 110 may generate the risk score 301 for a patient at the particular time stamp by aggregating each risk sub-score for the individual vital measurements at that time stamp. For example, in some embodiments, the score engine 110 determines a sum, or weighted sum, of the risk sub-scores. However, another example, the score engine 110 may determine an average or weighted average of the risk sub-scores. As a result, the score engine 110 generates a risk score 301, e.g., on a scale from 0 at the least risk and 15 at the greatest risk, for each patient at each time stamp of the vital measurement data 102.

In some embodiments, a prediction label generator 302 may then construct sub-sets of the vital measurement data 102 for use in training and testing by labeling windows of the vital measurement data 102 according to the risk scores 301. For example, the prediction label generator 302 may produce a label for each retrospective or prospective prediction period based on the windows formed by the windowing engine 120 described above. In some embodiments, the prediction periods include, e.g., overnight periods such as, e.g., time stamps occurring between the hours of 10:00 PM and 6:00 AM, or other overnight period. Each prediction period may be labeled according to the greatest risk score 301 determined for that period, where each prediction period having a risk score greater than or equal to, e.g., 5, 6, or 7 on a scale of 0 to 15, may be labelled as high risk, thus indicating a need for increased monitoring. Meanwhile, each prediction period having a risk score less than, e.g., 5, 6, or 7 on a scale of 0 to 15, may be labelled as low risk, thus indicating decreased monitoring. As a result, ground truth data for each prediction period may be supplied to the optimizer 220 for assessing error in the corresponding predictions for the prediction periods.

In some embodiments, the stability prediction neural network 200 predicts the probability of a low risk label for each prediction period based on the vital measurement data 102 of non-prediction period data. In some embodiments, the non-prediction period vital measurement data 102 may be encoded by a data encoding 303 component. In some embodiments, the encoding may include any suitable data encoding technique for producing neural network input data, such as, e.g., one-hot encoding, however other encoding techniques are contemplated.

In some embodiments, the encoded vital measurement data 102 for non-prediction periods, e.g., preceding periods, may be ingested by the stability prediction neural network 200 in windows associated with preceding period for each prediction period, e.g., as windowed by the windowing engine 120 described above. Thus, sequences of encoded vital measurement data 102 may be used to generate predictions utilizing the stability prediction neural network 200, where each sequence spans a period of time preceding a prediction period, e.g., a period of time before, e.g., 10:00 PM, or before a night shift begins, e.g., before 6:00 PM or 8:00 PM or other suitable time. Thus, all vital measurements for a patient having time stamps preceding the prediction period may form the sequence of encoded vital measurement data 102 for that prediction period for that patient. In some embodiments, preceding period may include, e.g., about 5 hours, 6 hours, 8 hours, or 12 hours preceding the prediction period or preceding the night shift, or other suitable time.

In some embodiments, the stability prediction neural network 200 may generate a probability 304 for each patient for a prediction period based on the ingested encoded vital measurement data 102 of the preceding period preceding the prediction period. In some embodiments, the probability 304 for each prediction period indicates a predicted probability that the associated patient is a low risk patient, e.g., a patient that will have a risk score during the prediction period below, e.g., 5, 6 or 7 on a scale between 0 and 15.

In some embodiments, the probability 304 for each prediction period for each patient may be received by the optimizer 220 and compared against the corresponding labels for the prediction period produced by the prediction label generator 302. As described above, the optimizer 220 may employ a suitable objective function, such as, e.g., cross-entropy loss, to determine an error of the probabilities 304 according to the labels. In some embodiments, based on the error determined by the optimizer 220, the optimizer 220 may backpropagate the error to the stability prediction neural network 200 to update weights of, e.g., the LSTM layers using a suitable backpropagation algorithm, such as, stochastic gradient descent, an Adam optimizer, AdaGrad, RMSProp, among others. In some embodiments, the stability prediction neural network 200 may be re-trained on, e.g., a nightly, a weekly, or a monthly basis, or according to any other re-training schedule with additional vital measurement data 102.

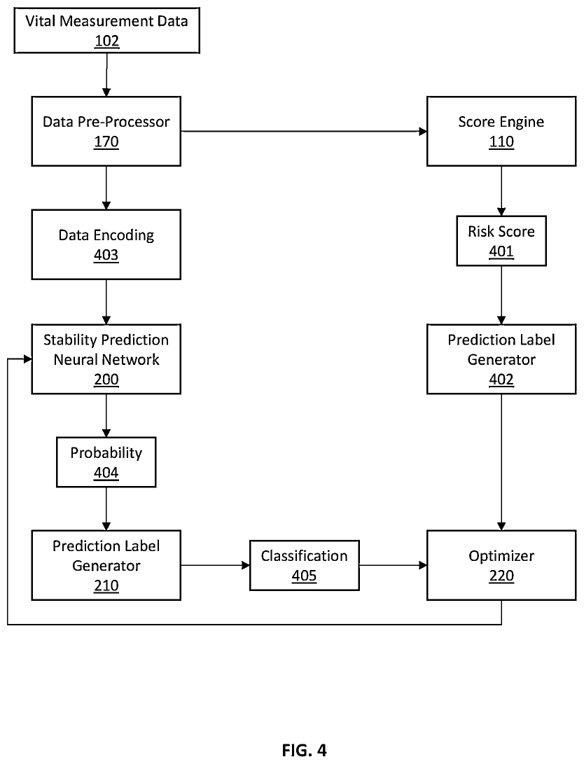

FIG. 4 illustrates a flowchart of another illustrative methodology for training a stability prediction neural network to predict condition measurements to automatically establish patient care schedules in accordance with one or more embodiments of the present disclosure.

In some embodiments, the data pre-processor 170 receives sequences of the vital measurement data 102 for each patient of the healthcare facility 101. In some embodiments, the vital measurement data 102 include sequences of, e.g., respiration rate measurements, heart rate measurements, blood pressure measurements, temperature measurements, among others. The data pre-processor 170 may cleanse and normalize the sequences of the vital measurement data 102, such as, e.g., removing outliers, imputing missing measurement (e.g., from a temporally near measurements, such as within about 12 hours of the missing measurement), among other pre-processing tasks.

In some embodiments, a dataset for training and testing the stability prediction neural network 200 may be constructed using the sequences of vital measurement data 102 for each patient. To do so, a ground truth dataset is built, whereby vital measurement data 102 for prediction periods is provided to the score engine 110. As described above, the vital measurement data 102 of the prediction periods may be extracted by the windowing engine 120 according to vital measurement time stamps being within an overnight period, such as between the hours of about 10:00 PM and 6:00 AM. In some embodiments, the score engine 110 may generate risk scores 401 at each time stamp of measurements in the sequence during the prediction periods. To produce the risk scores 401, the score engine 110 may calculate a sub-score for each vital measurement at the time stamp for each patient. For example, where the vital measurement data 102 includes respiration rate measurements, heart rate measurements, blood pressure measurements, and temperature measurements at each time stamp, the score engine 110 may calculate a risk sub-score for respiration rate at a particular time stamp, a risk sub-score for heart rate at the particular time stamp, and a risk sub-score for temperature measurement at the particular time stamp. In some embodiments, each sub-score may be indicative of a patient condition or risk of deterioration at the particular time stamp.

In some embodiments, the score engine 110 may generate the risk score 401 for a patient at the particular time stamp by aggregating each risk sub-score for the individual vital measurements at that time stamp. For example, in some embodiments, the score engine 110 determines a sum, or weighted sum, of the risk sub-scores. However, another example, the score engine 110 may determine an average or weighted average of the risk sub-scores. As a result, the score engine 110 generates a risk score 401, e.g., on a scale from 0 at the least risk and 15 at the greatest risk, for each patient at each time stamp of the vital measurement data 102.

In some embodiments, a prediction label generator 402 may then construct sub-sets of the vital measurement data 102 for use in training and testing by labeling windows of the vital measurement data 102 according to the risk scores 401. For example, the prediction label generator 402 may produce a label for each retrospective or prospective prediction period based on the windows formed by the windowing engine 120 described above. In some embodiments, the prediction periods include, e.g., overnight periods such as, e.g., time stamps occurring between the hours of 10:00 PM and 6:00 AM, or other overnight period. Each prediction period may be labeled according to the greatest risk score 401 determined for that period, where each prediction period having a risk score greater than or equal to, e.g., 5, 6, or 7 on a scale of 0 to 15, may be labelled as high risk, thus indicating a need for increased monitoring. Meanwhile, each prediction period having a risk score less than, e.g., 5, 6, or 7 on a scale of 0 to 15, may be labelled as low risk, thus indicating decreased monitoring. As a result, ground truth data for each prediction period may be supplied to the optimizer 220 for assessing error in the corresponding predictions for the prediction periods.

In some embodiments, the stability prediction neural network 200 predicts the probability of a low risk label for each prediction period based on the vital measurement data 102 of non-prediction period data. In some embodiments, the non-prediction period vital measurement data 102 may be encoded by a data encoding 403 component. In some embodiments, the encoding may include any suitable data encoding technique for producing neural network input data, such as, e.g., one-hot encoding, however other encoding techniques are contemplated.

In some embodiments, the encoded vital measurement data 102 for non-prediction periods, e.g., preceding periods, may be ingested by the stability prediction neural network 200 in windows associated with preceding period for each prediction period, e.g., as windowed by the windowing engine 120 described above. Thus, sequences of encoded vital measurement data 102 may be used to generate predictions utilizing the stability prediction neural network 200, where each sequence spans a period of time preceding a prediction period, e.g., a period of time before, e.g., 10:00 PM, or before a night shift begins, e.g., before 6:00 PM or 8:00 PM or other suitable time. Thus, all vital measurements for a patient having time stamps preceding the prediction period may form the sequence of encoded vital measurement data 102 for that prediction period for that patient. In some embodiments, preceding period may include, e.g., about 5 hours, 6 hours, 8 hours, or 12 hours preceding the prediction period or preceding the night shift, or other suitable time.

In some embodiments, the stability prediction neural network 200 may generate a probability 404 for each patient for a prediction period based on the ingested encoded vital measurement data 102 of the preceding period preceding the prediction period. In some embodiments, the probability 404 for each prediction period indicates a predicted probability that the associated patient is a low risk patient, e.g., a patient that will have a risk score during the prediction period below, e.g., 5, 6 or 7 on a scale between 0 and 15.

In some embodiments, the probability 404 for each prediction period for each patient may be received by the prediction label generator 210. In some embodiments, the prediction label generator 210 implements a thresholding mechanism that compares the probability 404 predicted for each prediction period for each patient against a probability threshold. In some embodiments, the probability threshold may be predetermined to balance false positives against false negatives. In some embodiments, a false positive is a patient that is predicted to be stable during a period of time, but actually was not stable. In some embodiments, a false negative is a patient predicted to be unstable during a period of time, but actually was not unstable. Weighing false positives as more serious errors make only sense in the context of the “letting patients sleep” scheme, In some embodiments, a negative prediction (e.g., a patient will be unstable) may result in the default course of care of vital measurements, while a positive prediction (e.g., a patient will be stable) results in a reduced vital measurement schedule to facilitate improved sleep. However, a false positive has a greater degree of a risk of harm than a false negative in this context, thus predictions are thresholded to prevent high false positives that would incorrectly predict stable patients. Accordingly, the prediction threshold may be any suitable threshold on the predicted probability, such as, e.g., about 0.95, about 0.99, 0.99095, or other threshold. In some embodiments, any suitable threshold may be used, e.g., to achieve a 95% confidence interval that fewer than, e.g., 2 in 10,000 prediction periods are falsely predicted to be stable (e.g., false positive predictions). Thus, probabilities 404 above the prediction threshold are classified by the prediction label generator as low risk of deterioration during the prediction period thus indicating a need for decreased monitoring during the prediction, while probabilities 404 below the prediction threshold are classified as high risk, thus indicating a need for greater monitoring. Accordingly, each preceding period of encoded vital measurement data 102 sequences is used to produce a classification 405 for a prediction period for each patient subsequent to each preceding period.

In some embodiments, the optimizer 220 receives the classifications 405 and compares the classifications 405 against the corresponding labels for the prediction period produced by the prediction label generator 402. As described above, the optimizer 220 may employ a suitable objective function, such as, e.g., binary cross-entropy loss, to determine an error of the probabilities 404 according to the labels. In some embodiments, the binary cross-entropy loss is used to produce a probability measure that may be used to threshold the confidence interval, thus enabling fine tuning of the amount of false positives with a single threshold. In some embodiments, based on the error determined by the optimizer 220, the optimizer 220 may backpropagate the error to the stability prediction neural network 200 to update weights of, e.g., the LSTM layers using a suitable backpropagation algorithm, such as, stochastic gradient descent, an Adam algorithm, AdaGrad, RMSProp, among others. In some embodiments, the stability prediction neural network 200 may be re-trained on, e.g., a nightly, a weekly, or a monthly basis, or according to any other re-training schedule with additional vital measurement data 102.

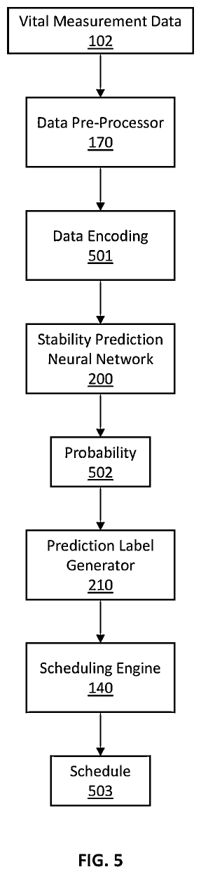

FIG. 5 illustrates a flowchart of an illustrative methodology for predicting condition measurements with a stability prediction neural network to automatically establish patient care schedules in accordance with one or more embodiments of the present disclosure.

In some embodiments, the data pre-processor 170 receives sequences of the vital measurement data 102 for each patient of the healthcare facility 101. In some embodiments, the vital measurement data 102 include most recent sequences of, e.g., respiration rate measurements, heart rate measurements, blood pressure measurements, temperature measurements, among others. The data pre-processor 170 may cleanse and normalize the sequences of the vital measurement data 102, such as, e.g., removing outliers, imputing missing measurement (e.g., from a temporally near measurements, such as within about 12 hours of the missing measurement), among other pre-processing tasks.

In some embodiments, the stability prediction neural network 200 predicts the probability of a low risk label for a prediction period of an upcoming overnight period following the most recent time stamp of the vital measurement data 102. In particular, the trained stability prediction neural network may generate a prediction for the next prediction period based on the vital measurement data 102 of non-prediction period data. In some embodiments, the vital measurement data 102 may be encoded by a data encoding 501 component. In some embodiments, the encoding may include any suitable data encoding technique for producing neural network input data, such as, e.g., one-hot encoding, however other encoding techniques are contemplated.

In some embodiments, the encoded vital measurement data 102 may be ingested by the stability prediction neural network 200 in a window associated with a most recent preceding period window of vital measurement data 102, e.g., as windowed by the windowing engine 120 described above. Thus, the sequences of encoded vital measurement data 102 may be used to generate predictions utilizing the stability prediction neural network 200, where each sequence spans a period of time preceding the upcoming prediction period, e.g., before 10:00 PM, or before a night shift begins, e.g., before 6:00 PM or 8:00 PM or other suitable time. Thus, all vital measurements for a patient having time stamps preceding the prediction period may form the sequence of encoded vital measurement data 102 for that prediction period for that patient. In some embodiments, preceding period may include, e.g., about 5 hours, 6 hours, 8 hours, or 12 hours preceding the prediction period or preceding the night shift, or other suitable time.

In some embodiments, the stability prediction neural network 200 may generate a probability 502 for each patient for a prediction period based on the ingested encoded vital measurement data 102. In some embodiments, the probability 502 for the prediction period indicates a predicted probability that the associated patient is a low risk patient, e.g., a patient that will have a risk score during the prediction period below, e.g., 5, 6 or 7 on a scale between 0 and 15.

In some embodiments, the probability 502 for the prediction period for each patient may be received by the prediction label generator 210. In some embodiments, the prediction label generator 210 implements a thresholding mechanism that compares the probability 502 for each patient against a probability threshold. In some embodiments, the probability threshold may be predetermined to balance false positives against false negatives, where a false positive of a low risk patient during a prediction period is less acceptable than a false negative. Accordingly, the prediction threshold may be, e.g., about 0.99095 to achieve a 95% confidence interval that fewer than, e.g., 2 in 10,000 prediction periods receive a false positive label of low risk. Thus, probabilities 502 above, e.g., about 0.99095 are classified by the prediction label generator as low risk of deterioration during the prediction period thus indicating a need for decreased monitoring during the prediction, while probabilities 502 below 0.99095 are classified as high risk, thus indicating a need for greater monitoring. Accordingly, the sequences of encoded vital measurement data 102 are used to produce a classification for the prediction period for each patient to predict whether each patient will be high risk or low risk of deterioration.

In some embodiments, the scheduling engine 140 may receive the classification for each patient and automatically adjust patient monitoring schedules 503. Where the classifications indicate that a patient will experience high instability, the scheduling engine 140 may automatically select a high risk schedule for the patient. In some embodiments, a high risk schedule may include patient monitoring sessions, e.g., every hour, every two hours, every three hours, or every four hours. However, where the classifications indicate a low risk of instability or condition deterioration, the scheduling engine 140 may automatically select a low risk schedule for the patient. For example, a low risk schedule may include, e.g., only one monitoring session overnight, Thus, facilitating improved sleep for the low risk patient, thus improving patient outcomes.

In some embodiments, the scheduling engine 140 may automatically apply the selected patient monitoring schedule for each patient to an electronic rounds schedule of one or more healthcare personnel assigned to each patient, such as an electronic calendar. Accordingly, the scheduling engine 140 may automatically program an electronic schedule of each healthcare personnel with the rounds schedules. In some embodiments, the scheduling engine 140 may additionally or alternatively instruct a printer to print the selected patient monitoring schedule for each patient, or store the patient monitoring schedules for each patient for access by the user computing devices 105, or both.

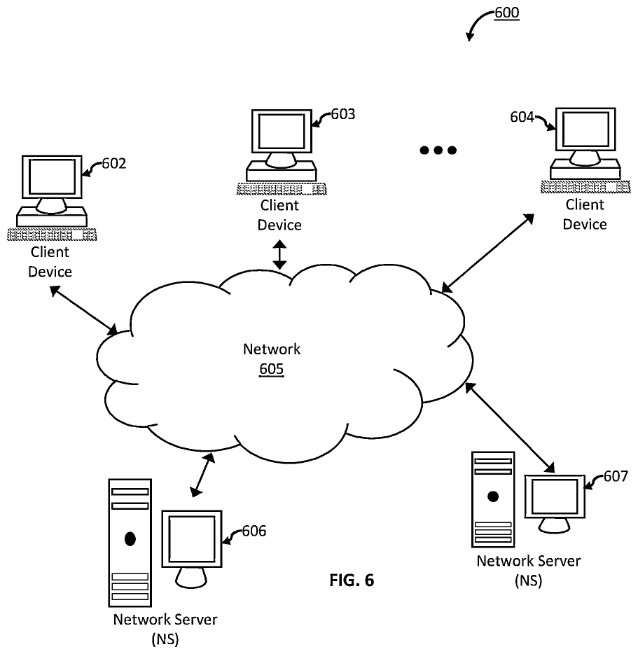

FIG. 6 depicts a block diagram of an exemplary computer-based system and platform 600 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 600 may be configured to manage a large number of members and concurrent transactions, as detailed herein. In some embodiments, the exemplary computer-based system and platform 600 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 6, members 602-704 (e.g., clients) of the exemplary computer-based system and platform 600 may include virtually any computing device capable of receiving and sending a message over a network (e.g., cloud network), such as network 605 including connect vital measurement devices in a healthcare facility, to and from another computing device, such as servers 606 and 607, each other, and the like. In some embodiments, the member devices 602-704 may be vital measurement devices, personal computers, multiprocessor systems, microprocessor-based or programmable consumer electronics, network PCs, and the like. In some embodiments, one or more member devices within member devices 602-704 may include computing devices that typically connect using a wireless communications medium such as cell phones, smart phones, internet-of-things devices, integrated devices combining one or more of the preceding devices, or virtually any mobile computing device, and the like. In some embodiments, one or more member devices within member devices 602-704 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, satellite, ZigBee, etc.). In some embodiments, one or more member devices within member devices 602-704 may include may run one or more applications, such as Internet browsers, mobile applications, voice calls, video games, videoconferencing, and email, among others. In some embodiments, one or more member devices within member devices 602-704 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 602-704 may be specifically programmed by either Java, .Net, QT, C, C++ and/or other suitable programming language. In some embodiments, one or more member devices within member devices 602-704 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 605 may provide network access, data transport and/or other services to any computing device coupled to it. In some embodiments, the exemplary network 605 may include and implement at least one specialized network architecture that may be based at least in part on one or more standards set by, for example, without limitation, Global System for Mobile communication (GSM) Association, the Internet Engineering Task Force (IETF), 4G LTE, 5G, and the Worldwide Interoperability for Microwave Access (WiMAX) forum. In some embodiments, the exemplary network 605 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 605 may include and implement, as an alternative or in conjunction with one or more of the above, a WiMAX architecture defined by the WiMAX forum. In some embodiments and, optionally, in combination of any embodiment described above or below, the exemplary network 605 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 605 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 605 may also include mass storage, such as network attached storage (NAS), a storage area network (SAN), a content delivery network (CDN) or other forms of computer or machine readable media.

In some embodiments, the exemplary server 606 or the exemplary server 607 may be a web server (or a series of servers) running a network operating system, examples of which may include but are not limited to, cloud platforms, Microsoft Windows Server, Novell NetWare, or Linux. In some embodiments, the exemplary server 606 or the exemplary server 607 may be used for and/or provide cloud and/or network computing. Although not shown in FIG. 6, in some embodiments, the exemplary server 606 or the exemplary server 607 may have connections to external systems like email, SMS messaging, text messaging, ad content providers, etc. Any of the features of the exemplary server 606 may be also implemented in the exemplary server 607 and vice versa.

In some embodiments, one or more of the exemplary servers 606 and 607 may be specifically programmed to perform, in non-limiting example, as authentication servers, search servers, email servers, social networking services servers, SMS servers, IM servers, MMS servers, exchange servers, photo-sharing services servers, advertisement providing servers, financial/banking-related services servers, travel services servers, or any similarly suitable service-base servers for users of the member computing devices 601-704.

In some embodiments and, optionally, in combination of any embodiment described above or below, for example, one or more exemplary computing member devices 602-704, the exemplary server 606, and/or the exemplary server 607 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

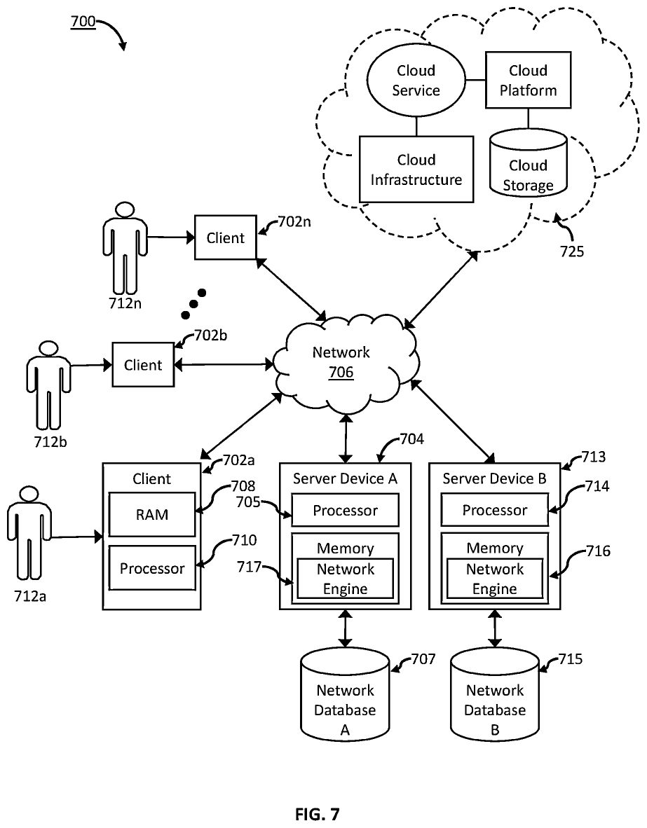

FIG. 7 depicts a block diagram of another exemplary computer-based system and platform 700 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the member computing devices 702a, 702b thru 702n shown each at least includes a computer-readable medium, such as a random-access memory (RAM) 708 coupled to a processor 710 or FLASH memory. In some embodiments, the processor 710 may execute computer-executable program instructions stored in memory 708. In some embodiments, the processor 710 may include a microprocessor, an ASIC, and/or a state machine. In some embodiments, the processor 710 may include, or may be in communication with, media, for example computer-readable media, which stores instructions that, when executed by the processor 710, may cause the processor 710 to perform one or more steps described herein. In some embodiments, examples of computer-readable media may include, but are not limited to, an electronic, optical, magnetic, or other storage or transmission device capable of providing a processor, such as the processor 710 of client 702a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, member computing devices 702a through 702n may also comprise a number of external or internal devices such as a mouse, a CD-ROM, DVD, a physical or virtual keyboard, a display, or other input or output devices. In some embodiments, examples of member computing devices 702a through 702n (e.g., clients) may be any type of processor-based platforms that are connected to a network 706 such as, without limitation, personal computers, digital assistants, personal digital assistants, smart phones, pagers, digital tablets, laptop computers, Internet appliances, and other processor-based devices. In some embodiments, member computing devices 702a through 702n may be specifically programmed with one or more application programs in accordance with one or more principles/methodologies detailed herein. In some embodiments, member computing devices 702a through 702n may operate on any operating system capable of supporting a browser or browser-enabled application, such as Microsoft™, Windows™, and/or Linux. In some embodiments, member computing devices 702a through 702n shown may include, for example, personal computers executing a browser application program such as Microsoft Corporation's Internet Explorer™, Apple Computer, Inc.'s Safari™, Mozilla Firefox, and/or Opera. In some embodiments, through the member computing client devices 702a through 702n, users, 712a through 702n, may communicate over the exemplary network 706 with each other and/or with other systems and/or devices coupled to the network 706. As shown in FIG. 7, exemplary server devices 704 and 713 may be also coupled to the network 706. In some embodiments, one or more member computing devices 702a through 702n may be mobile clients.

In some embodiments, at least one database of exemplary databases 707 and 715 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

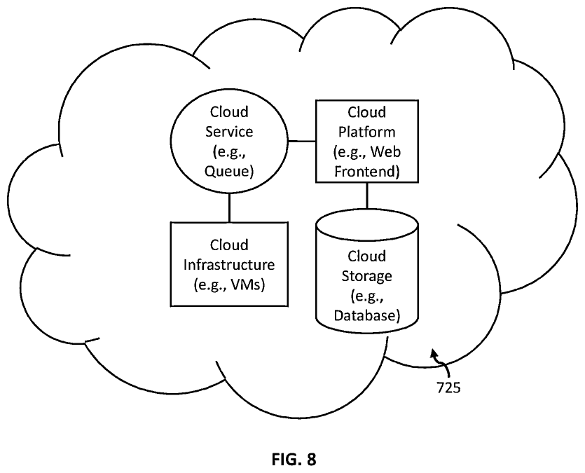

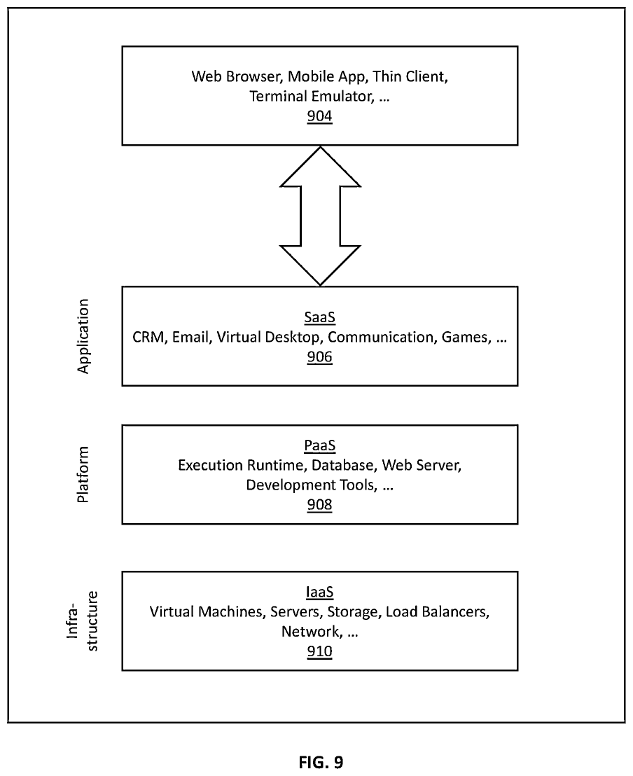

In some embodiments, the exemplary inventive computer-based systems/platforms, the exemplary inventive computer-based devices, and/or the exemplary inventive computer-based components of the present disclosure may be specifically configured to operate in a cloud computing/architecture 725 such as, but not limiting to: infrastructure a service (IaaS) 910, platform as a service (PaaS) 908, and/or software as a service (SaaS) 906 using a web browser, mobile app, thin client, terminal emulator or other endpoint 904. FIGS. 8 and 9 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

FIGS. 10-24 illustrative results of an embodiment of the stability prediction system in accordance with aspects of the present invention.

In some embodiments, the model engine 130 employs a sequence of prior vital measurement data 102 for a given patient and predicts the probability 304 of the patient's deterioration for any given night, in order to determine the necessity of assessing overnight vitals. The algorithm ingests the sequence of prior vital measurement data 102, such as breathing rate (BR), heart rate (HR), blood pressure (BP) and temperature, as well as the patient's age and a Modified Early Warning (MEWS) risk score. FIG. 10 provides an example of a sequence of samples as input, each sample containing four vital signals and other input variables not depicted here, such as hour of the day, the time since previous sample, the change in risk score and the variance of risk sub-scores. The green band signifies the normal range of values. Nights (purple band) are labelled according to the risk score values they enclose: if any of the scores exceeds a certain threshold, the patient is to be woken up for vital recording (potentially unstable case), otherwise the patient may sleep.

In some embodiments, the specific risk score 301, calculated with a formula by the scoring engine 110 from the vital measurement data 102, produces a risk score 304 on a scale from 0 to 15, with risk scores 304 approaching 15 indicating instability in the patient's medical state and hence compels a more strenuous care involving frequent vital recordings. In some embodiments, a patient is deemed at overnight risk of deterioration when their score 301 throughout the night becomes equal to or exceeds 7, as calculated from vitals taken any time during the night, e.g., after 10:00 PM and before 6:00 AM. As shown in the example of FIG. 10, vital measurements are plotted against the time of the day they were taken. Based on the formula of the risk score 301 calculation, certain ranges of values for the different vitals can be deemed as normal (not adding any values to the risk score).

In some embodiments, the stability prediction neural network 200 is trained to predict the risk of deterioration for any night, using a sequence of prior vital records during the hospital stay of each patient. The retrospective data used to create the stability prediction neural network 200 is split into training, validation and test data, e.g., by the data pre-processor 170.

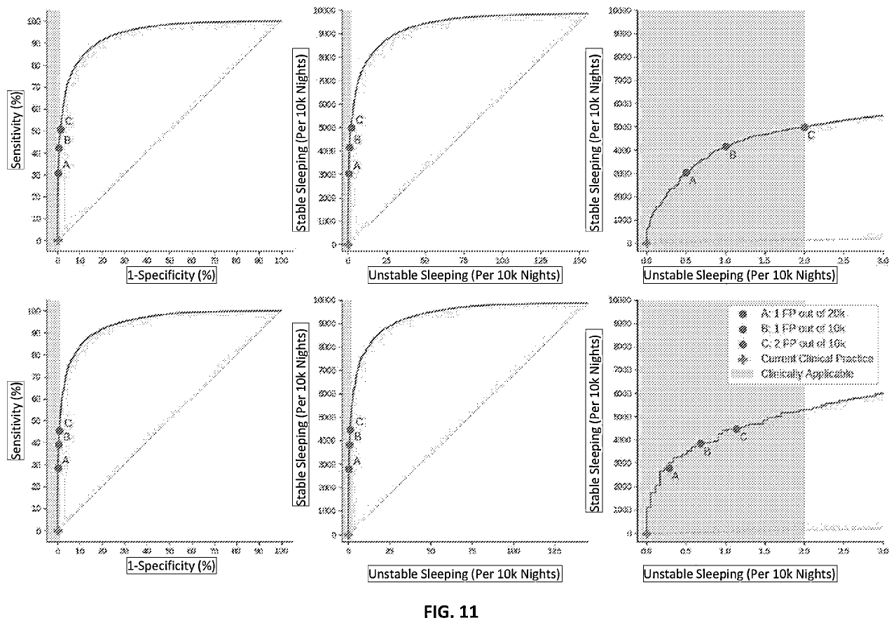

In some embodiments, for both retrospective and prospective test datasets, the stability prediction neural network 200 outputs a probability 304 value between 0 and 1 for each prediction. This value is proportionate to the stability prediction neural network 200 estimate of the probability that the patient will be stable overnight. To determine the stability prediction neural network 200 binary suggestion of whether overnight vitals should be taken, a threshold to this probability has to be chosen. In some embodiments, to assess the efficacy of the stability prediction neural network 200, receiver operating curves (ROC) are constructed for both retrospective and prospective datasets using multiple thresholds, where true positives (TP) correspond to the percentage of nights of patient left sleeping safely and false positives (FP) to nights of patients left sleeping while becoming potentially unstable (risk score 301 of greater than or equal to 7), as shown in FIG. 11. Due to the nature of the predictions of the stability prediction neural network 200, the rate of false positives needs to be very low and the stability prediction neural network 200 needs to be conservative in predicting a safe patient night. Based on feedback from multiple health provider collaborators, the clinically applicable rate of missed detections (patients sleeping that are at risk of deterioration) may be selected to be at most, e.g., 2 out of 10,000 patient nights. Based on this feedback, it became apparent that a more clinically relevant variant of the ROC curve would juxtapose the number of true and false positives normalized by 10,000 nights; i.e. TP_{clin}=TP/(P+N) and FP_{clin}=FP/(P+N), where TP_{clin} and FP_{clin} are the clinically relevant True and False Positives respectively, TP and FP are the previously mentioned True Positives and False Positives, P represents the Positive and N the Negative cases.

FIG. 11 depicts the ROC curves of an embodiments of the stability prediction neural network 200, where the X axis represents the number of patient nights (out of 10000) where the patients were left sleeping while having an elevated risk of short term decompensation. The Y axis represents the number of patient nights (out of 10000) where the patients were left sleeping appropriately to their risk level. Dots represent three different model thresholds, which may be chosen according to the number of false positives they yielded for the retrospective test set. For instance, the operating threshold for model C is selected to keep the number of unstable sleeping nights below 2 out of 10,000, which is at the edge of the clinically applicable region described above. The same model thresholds were used in prospective testing (d, e, f).

Once the stability prediction neural network 200 is trained and the ROC curves were constructed for both retrospective and prospective test datasets, 3 different confidence thresholds may be established (A, B and C, of FIG. 11), out of which the least conservative, threshold C, can avoid overnight vitals for approximately 50% of the patient nights, while only missing suggesting vitals assessment in 2 out of 10,000 nights. An area under the receiver operating characteristic curve of 93.84% (95% confident interval (CI) 93.79-93.88%) is achieved on the retrospective, and 94.53% (95% CI 94.48-94.58%) is achieved on the prospective test sets. As shown in FIG. 11, the clinically applicable region is established for this particular problem at 2 false positive predictions per 10,000 nights (1.29% false positive rate), with the primary model threshold, C, lying on the edge of this region. Thresholds A and B correspond to even more conservative models, with false positive rates of 0.32% and 0.65%, respectively.

In some embodiments, the stability prediction neural network 200 may be constructed, trained and tested with two more different risk score cutoffs (>5 and >6), corresponding to even more conservative risk score policies. The ROC curves and clinically relevant ROC curves show that their performance is comparable to the stability prediction neural network 200 using the previously chosen risk score cutoff of 7 or greater, indicating the robustness of this approach to different levels of risk aversion from health systems and health providers.

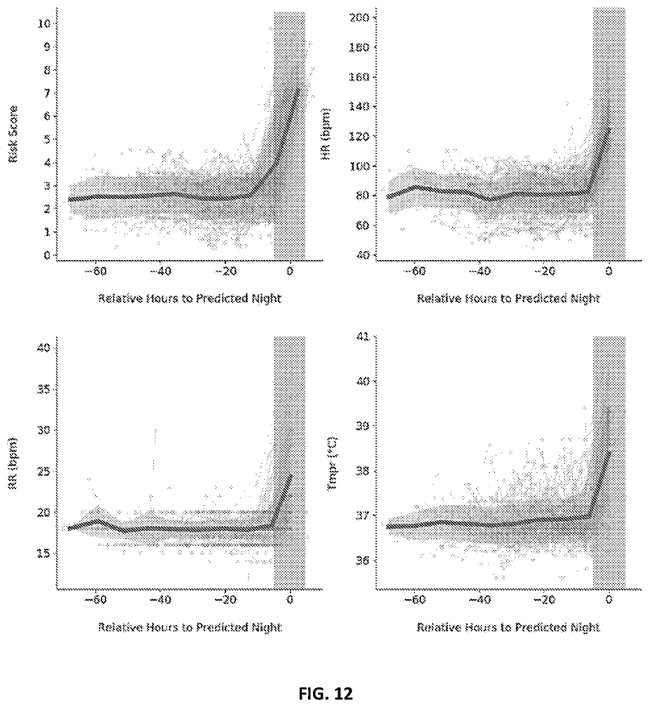

While the stability prediction neural network 200 is designed to be conservative and minimize the number of potentially unstable patient nights, in retrospective datasets it falsely classified 132 patient nights as overnight stable. To further understand the limitations of the stability prediction neural network 200 through the characteristics of these falsely classified cases, the trajectories of risk scores and vitals preceding the false positive nights were inspected, for a time window of 3 days. As illustrated in FIG. 12, the risk score, respiratory rate, temperature and heart rate for these cases stay mostly steady throughout the 3-day period and abruptly increase during the falsely classified night, outlining the absence of any visible trend that could potentially inform a predictive model.

FIG. 12 depicts trajectories of risk score and vital measurements preceding missed, false positive patient nights. False positive trajectories of risk score and vital signals stay flat before, and jumps abruptly on the missed night. The darker line represents the mean trajectory, the lighter band covers two standard deviations, the vertical band demarcates the missed night.

FIG. 13 depicts the differences in the nighttime vital measurements between the stable and unstable sleeping scenarios (A). During the majority of the missed 132 nights, risk scores did not exceed 7, and in only two instances reached the score 10 out of 15. Vital records measured during stable (true positive) and potentially unstable (false positive) were compared. All (B) HR, (C) RR, and (D) Tmpr recordings were found to be significantly higher in the unstable case.

While the history of the risk score and the trends are not informative enough to guide the stability prediction neural network 200, the severity of the falsely classified cases, as well as the specific differences in risk scores and overnight vitals between the cases were identified where the stability prediction neural network 200 correctly predicted stable patient nights and the incorrectly identified as stable, but potentially unstable patient nights. As shown in FIG. 13, in the falsely classified cases, the risk scores barely exceeded the chosen threshold of 7, with only 2 cases reaching a maximum risk score of 10 out of 15. This distribution of risk scores points out that even in the rare cases when patient nights are misclassified as stable, they mostly correspond to marginally unstable patient nights. Moreover, HR, RR and temperature were higher in the incorrectly identified cases. More specifically, the respiratory rate (FIG. 13 (C)) of the patients during the misclassified nights (M=22.02, SD=6.32) was higher (unpaired t-test, p<0.001) than for the stable sleeping patients (M=17.56, SD=1.39). Similarly, the HR (FIG. 13 (B)) during misclassified nights (M=108.32, SD=27.75) exceeded (unpaired t-test, p<0.001) the HR of patients sleeping stable (M=78.30, SD=13.36). Finally, body temperature (FIG. 13 (D)) during misclassified nights (M=37.64, SD=1.13) was higher (unpaired t-test, p<0.001) than for stable patients (M=36.81, SD=0.41), however the difference was not as pronounced as with the HR or RR. The values of HR and BR in the misclassified as stable cases hint that in most of them, these patients would not be able to sleep soundly, even with vital assessment skipped. Moreover, the differences between correctly and incorrectly classified cases show that HR and BR are the two principal discriminants, and could be used to this end if they could be tracked without obstructing the patients' sleep.

FIG. 14 depicts live detection of potentially unstable nights by simple HR thresholding. After the initial prediction made, patients who are let sleep may be further monitored, measuring only HR during the night. A patient exceeding a given HR threshold is suggested to be woken up for a proper vital recording. (A) Ratio of patient nights disrupted at different HR thresholds. Thresholds of 120 and 110 bpm were selected for further analysis according to the tradeoff between waking up stable and potentially unstable patients. (B) After HR thresholding at the selected levels, risk scores of 10 are detected and a decrease in the number of unstable patient nights at all risk scores is indicated.

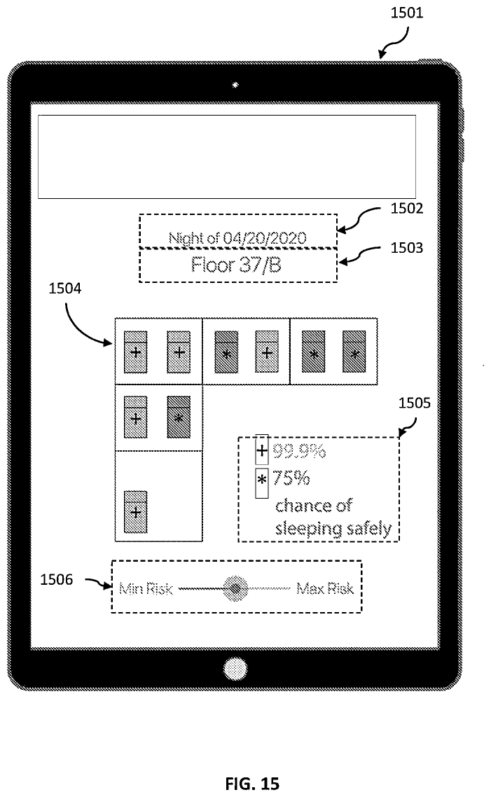

FIG. 15 depicts a user interface of a user device 1501 for displaying patient monitoring alerts and sleeping schedule modification recommendations based on a prediction of a risk of deterioration while asleep. In some embodiments, the user interface may include a depiction of occupied beds 1504 in the healthcare facility for a given night 1502 at a given location 1503. For example, the user interface may show the beds 1504 for Floor 37B on Apr. 20, 2020, as shown in FIG. 15. Each depicted bed 1504 may be color coded based on the prediction by the stability prediction neural network where, e.g., red or some other color or shading may indicate a certain risk of patient deterioration overnight, such as, e.g., a greater than 99 percent chance of remaining stable, or less than a 75 percent chance of remaining stable. However, any suitable values of the probability of remaining stable or deteriorating may be used.

In some embodiments, the color coding may be defined in a key 1505 to indicate which color or shading refers to a low risk and which color or shading refers to a not low risk. In some embodiments, the key 1505 may adjust based on user defined risk thresholds, e.g., using a risk adjustment interface element 1506, such as slider, toggle, text input, or other interface element. In some embodiments, adjusting risk with the risk adjustment interface element 1506 may cause the value in the key 1504 indicating high risk to adjust in accordance with the user selection, the value in the key 1504 indicating low risk to adjust in accordance with the user selection, or both.

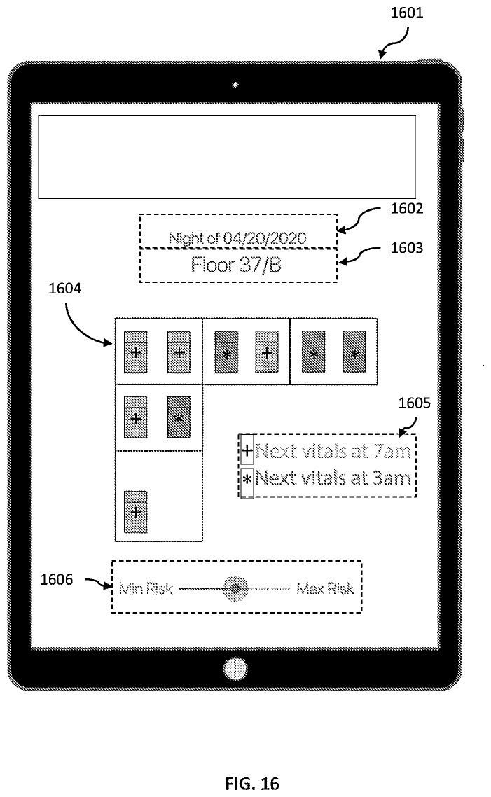

FIG. 16 depicts a user interface of a user device 1601 for displaying patient monitoring alerts and sleeping schedule modification recommendations based on a prediction of a risk of deterioration while asleep. In some embodiments, the user interface may include a depiction of occupied beds 1604 in the healthcare facility for a given night 1602 at a given location 1603. For example, the user interface may show the beds 1604 for Floor 37B on Apr. 20, 2020, as shown in FIG. 16. Each depicted bed 1604 may be color coded based on the prediction by the stability prediction neural network where, e.g., red or some other color or shading may indicate patient monitoring schedule modification, such as, e.g., a next vital measurement time based on each patient's probability of remaining stable or deteriorating overnight, such as, e.g., a next vital measurement time of 7:00 AM the following morning for low risk deterioration, and a next vital measurement time 3:00 AM for a high risk of deterioration.

In some embodiments, the color coding may be defined in a key 1605 to indicate which color or shading refers to a low risk schedule and which color or shading refers to a not low risk schedule. In some embodiments, the key 1605 may adjust based on user defined risk thresholds, e.g., using a risk adjustment interface element 1606, such as slider, toggle, text input, or other interface element. In some embodiments, adjusting risk with the risk adjustment interface element 1606 may cause the value in the key 1604 indicating a high risk schedule to adjust in accordance with the user selection, the value in the key 1604 indicating a low risk schedule to adjust in accordance with the user selection, or both.

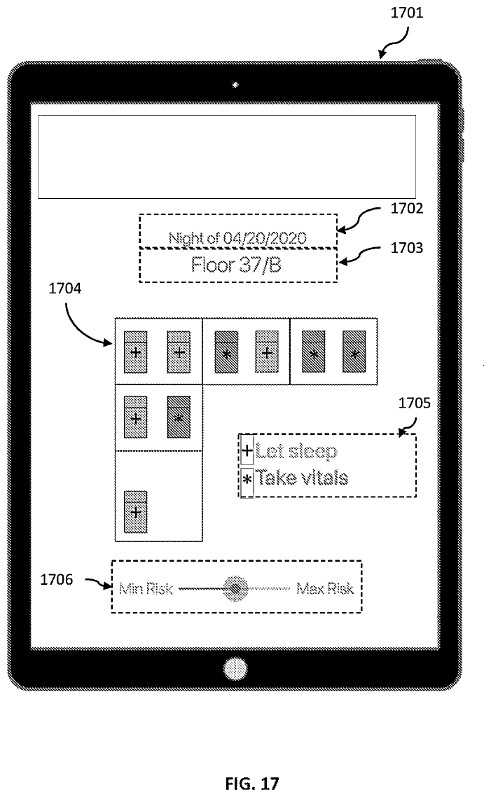

FIG. 17 depicts a user interface of a user device 1701 for displaying patient monitoring alerts and sleeping schedule modification recommendations based on a prediction of a risk of deterioration while asleep. In some embodiments, the user interface may include a depiction of occupied beds 1704 in the healthcare facility for a given night 1702 at a given location 1703. For example, the user interface may show the beds 1704 for Floor 37B on Apr. 20, 2020, as shown in FIG. 17. Each depicted bed 1704 may be color coded based on the prediction by the stability prediction neural network where, e.g., red or some other color or shading may indicate patient monitoring schedule directive, such as, e.g., to let the patient sleep or to wake the patient based on each patient's probability of remaining stable or deteriorating overnight.

In some embodiments, the color coding may be defined in a key 1705 to indicate which color or shading refers to a “let sleep” directive and which color or shading refers to a “wake” directive. In some embodiments, the key 1705 may adjust based on user defined risk thresholds, e.g., using a risk adjustment interface element 1706, such as slider, toggle, text input, or other interface element. In some embodiments, adjusting risk with the risk adjustment interface element 1706 may cause the value in the key 1704 indicating a “let sleep” directive to adjust in accordance with the user selection, the value in the key 1704 indicating a “wake” directive to adjust in accordance with the user selection, or both.

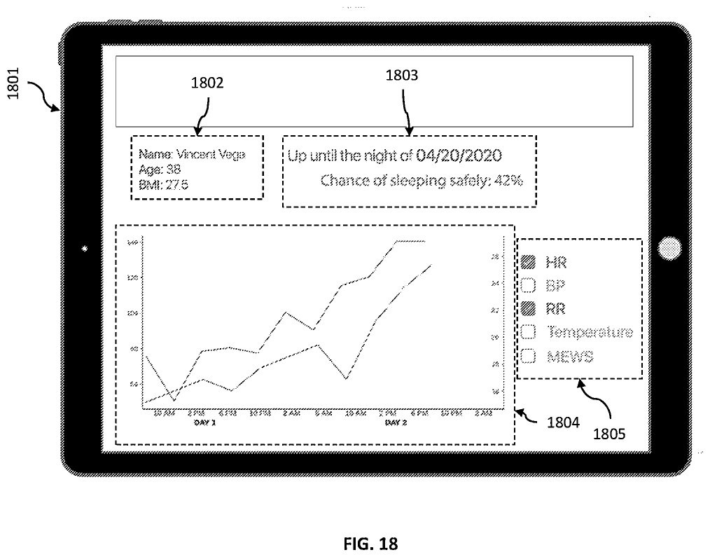

FIG. 18 depicts a user interface of a user device 1801 for displaying patient monitoring and sleeping schedule modifications for a selected patient. In some embodiments, a user may select a patient bed 1504, 1604 or 1704 from the user interface 1501, 1601 or 1701, respectively, or other suitable user interface for selecting patients. Upon selection, the user interface may include interface elements associated with the selected patient and sleep schedules for the patient based on risk of deterioration versus likelihood of remaining stable.

In some embodiments, the user interface may include a patient information component 1802 having patient information, such as, e.g., name, age, gender, medical condition, height, weight, body mass index (BMI), or other information and combinations thereof. In some embodiments, the patient information component 1802 may show the patient name, age and BMI for quick and easy identification by the user of the device 1801.

In some embodiments, the user interface may include condition assessment component 1803. In some embodiments, the condition assessment component 1803 includes information for the user to determine whether the patient is likely to sleep through the night with or without deterioration to the patient's condition. As such, the condition assessment component 1803 may assist the user to determine whether the patient is likely to sleep safely through the night with reduced monitoring. In some embodiments, the condition assessment component 1803 may display the condition assessment by indicating the date for the condition assessment or date of the most recent condition measurement, as well as a probability of sleeping safely through the night, e.g., as a percentage. In some embodiments, the percentage may be color coded to indicate whether the chance of sleeping safely indicates to let the patient sleep without monitoring or with reduced monitoring, or whether it indicates to monitor the patient during the night.

In some embodiments, the data in the condition assessment component 1803 may be informed by a history of patient vital measurements using the stability prediction neural network described above. In some embodiments, the history of measurements may be show in the user interface with a vitals history component 1804. In some embodiments, the vital history component 1804 may include a visualization of each vital measurement value in a given period, such as, e.g., for one day preceding the date of the condition assessment component 1803, one week preceding the date, one month preceding the date, or other period. In some embodiments, the vital measurements during the period may be depicted in the vitals history component 1804 as, e.g., one or more data plots, one or more line graphs, one or more bar graphs, one or more tables, or other visualization of the vital measurement data and combinations thereof.

In some embodiments, the vital measurement data of the vitals history component 1804 may be filterable with a vital measurement selection component 1805. In some embodiments, the vital measurement selection component 1805 may provide user selectable filters for, e.g., types of vital measurements to show in the vitals history component 1804, time periods of vital measurement data to show in the vitals history component 1804, or other filter on the vital measurement data. For example, the vital measurement selection component 1805 may provide interface elements for user selection of each vital measurement type of a selection of vital measurement types (e.g., heart rate, blood pressure, respiration rate, body temperature, MEWS score, or other vital measurement types). Thus, the user may select a desired vital or subset of vitals to view in the vitals history component 1805 to assist to inform whether to let the patient sleep without monitoring.

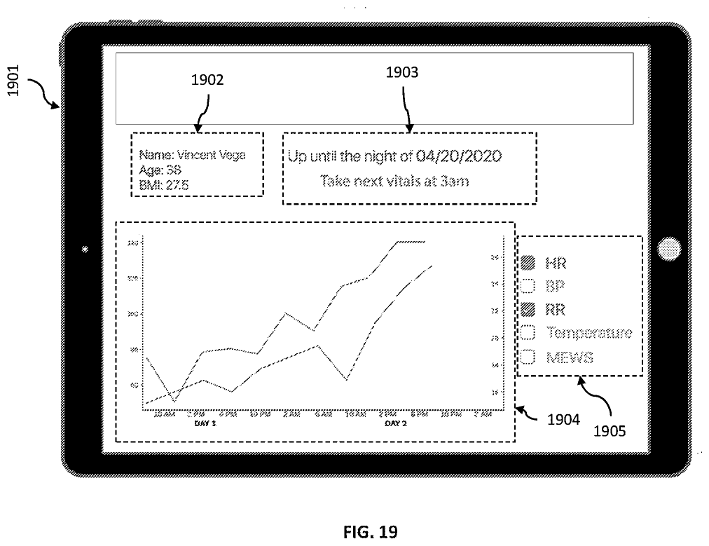

FIG. 19 depicts a user interface of a user device 1901 for displaying patient monitoring and sleeping schedule modifications for a selected patient. In some embodiments, a user may select a patient bed 1504, 1604 or 1704 from the user interface 1501, 1601 or 1701, respectively, or other suitable user interface for selecting patients. Upon selection, the user interface may include interface elements associated with the selected patient and sleep schedules for the patient based on risk of deterioration versus likelihood of remaining stable.

In some embodiments, the user interface may include a patient information component 1902 having patient information, such as, e.g., name, age, gender, medical condition, height, weight, body mass index (BMI), or other information and combinations thereof. In some embodiments, the patient information component 1902 may show the patient name, age and BMI for quick and easy identification by the user of the device 1901.

In some embodiments, the user interface may include a monitoring schedule component 1903. In some embodiments, the monitoring schedule component 1903 includes information for the user to determine whether the patient is likely to sleep through the night with or without deterioration to the patient's condition. As such, the monitoring schedule component 1903 may employ the predicted stability of the patient through the night to formulate and display a vitals monitoring schedule or schedule modification, such as a next time at which to wake the patient to monitor vitals (e.g., at 3 AM or not until 7 AM the following morning, or other schedule times).

In some embodiments, the schedule or schedule modification in the monitoring schedule component 1903 may be informed by a history of patient vital measurements using the stability prediction neural network described above. In some embodiments, the history of measurements may be show in the user interface with a vitals history component 1904. In some embodiments, the vital history component 1904 may include a visualization of each vital measurement value in a given period, such as, e.g., for one day preceding the date of the condition assessment component 1903, one week preceding the date, one month preceding the date, or other period. In some embodiments, the vital measurements during the period may be depicted in the vitals history component 1904 as, e.g., one or more data plots, one or more line graphs, one or more bar graphs, one or more tables, or other visualization of the vital measurement data and combinations thereof.

In some embodiments, the vital measurement data of the vitals history component 1904 may be filterable with a vital measurement selection component 1905. In some embodiments, the vital measurement selection component 1905 may provide user selectable filters for, e.g., types of vital measurements to show in the vitals history component 1904, time periods of vital measurement data to show in the vitals history component 1904, or other filter on the vital measurement data. For example, the vital measurement selection component 1905 may provide interface elements for user selection of each vital measurement type of a selection of vital measurement types (e.g., heart rate, blood pressure, respiration rate, body temperature, MEWS score, or other vital measurement types). Thus, the user may select a desired vital or subset of vitals to view in the vitals history component 1905 to assist to inform whether to let the patient sleep without monitoring.

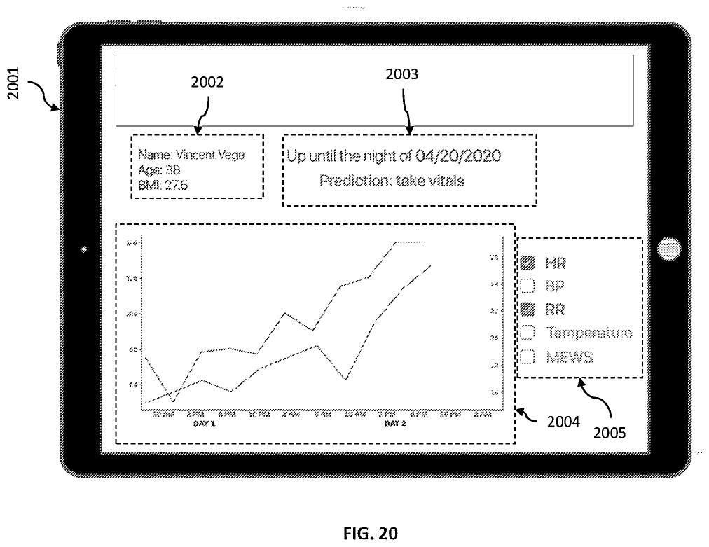

FIG. 20 depicts a user interface of a user device 2001 for displaying patient monitoring and sleeping schedule modifications for a selected patient. In some embodiments, a user may select a patient bed 1504, 1604 or 1704 from the user interface 1501, 1601 or 1701, respectively, or other suitable user interface for selecting patients. Upon selection, the user interface may include interface elements associated with the selected patient and sleep schedules for the patient based on risk of deterioration versus likelihood of remaining stable.

In some embodiments, the user interface may include a patient information component 2002 having patient information, such as, e.g., name, age, gender, medical condition, height, weight, body mass index (BMI), or other information and combinations thereof. In some embodiments, the patient information component 2002 may show the patient name, age and BMI for quick and easy identification by the user of the device 2001.

In some embodiments, the user interface may include a monitoring directive component 2003. In some embodiments, the monitoring directive component 2003 includes information for the user to determine whether the patient is likely to sleep through the night with or without deterioration to the patient's condition. As such, the monitoring directive component 2003 may employ the predicted stability of the patient through the night to formulate and display a vitals monitoring directive, such as whether to wake the patient to monitor vitals (e.g., “take vitals” or “let sleep” or other directive for whether or not to wake the patient or let sleep).

In some embodiments, the monitoring directive in the monitoring directive component 2003 may be informed by a history of patient vital measurements using the stability prediction neural network described above. In some embodiments, the history of measurements may be show in the user interface with a vitals history component 2004. In some embodiments, the vital history component 2004 may include a visualization of each vital measurement value in a given period, such as, e.g., for one day preceding the date of the condition assessment component 2003, one week preceding the date, one month preceding the date, or other period. In some embodiments, the vital measurements during the period may be depicted in the vitals history component 2004 as, e.g., one or more data plots, one or more line graphs, one or more bar graphs, one or more tables, or other visualization of the vital measurement data and combinations thereof.

In some embodiments, the vital measurement data of the vitals history component 2004 may be filterable with a vital measurement selection component 2005. In some embodiments, the vital measurement selection component 2005 may provide user selectable filters for, e.g., types of vital measurements to show in the vitals history component 2004, time periods of vital measurement data to show in the vitals history component 2004, or other filter on the vital measurement data. For example, the vital measurement selection component 2005 may provide interface elements for user selection of each vital measurement type of a selection of vital measurement types (e.g., heart rate, blood pressure, respiration rate, body temperature, MEWS score, or other vital measurement types). Thus, the user may select a desired vital or subset of vitals to view in the vitals history component 2005 to assist to inform whether to let the patient sleep without monitoring.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random-access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) Linux, (2) Microsoft Windows, (3) OS X (Mac OS), (4) Solaris, (5) UNIX (6) VMWare, (7) Android, (8) Java Platforms, (9) Open Web Platform, (10) Kubernetes or other suitable computer platforms. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

The aforementioned examples are, of course, illustrative and not restrictive.

At least some aspects of the present disclosure will now be described with reference to the following numbered clauses.

1. A method comprising:
receiving, by at least one processor, patient vital measurement data for each patient of a plurality of patients;
   wherein the each patient vital measurement data comprises a vital measurement time-sequence, comprising:
      i) respiration rate measurements,
      ii) heart rate measurements,
      iii) blood pressure measurements, and
      iv) temperature measurements;
determining, by the at least one processor, a sub-score for each vital measurement of the vital measurement sequence for each patient;
determining, by the at least one processor, a risk score for each vital measurement of the vital measurement sequence for each patient based on a sum of each sub-score for each vital measurement to produce a record of risk scores through time for each patient; receiving, by the at least one processor, a label for each predetermined time period of the vital measurement sequence based on a comparison of each risk score in each predetermined time period with a risk threshold;
   wherein the label comprises a low risk label where one or more risk scores of each vital measurement is below the risk threshold;
utilizing, by the at least one processor, a recurrent neural network to predict a probability of a low risk classification for each predetermined time period based at least in part on a subset of the plurality of vital measurements preceding each predetermined time period;
producing, by the at least one processor, a risk classification for each predetermined time period based on a respective probability of the low risk classification exceeding a classification probability threshold;
   wherein the risk classification comprises one of a low-risk classification and a not-low-risk classification;
determining, by the at least one processor, classification error based on a comparison of a respective risk classification and a respective label for each predetermined time period;
   wherein an incorrect low-risk classification is weighted eight times more than an incorrect not-low-risk classification; and
determining, by the at least one processor, updated weights of long short-term memory (LSTM) cell of the recurrent neural network based on the classification error to train the recurrent neural network.

2. A method comprising:
receiving, by at least one processor, patient vital measurement data for each patient of a plurality of patients;
   wherein the each patient vital measurement data comprises a vital measurement time-sequence, comprising:
      i) respiration rate measurements,
      ii) heart rate measurements,
      iii) blood pressure measurements, and
      iv) temperature measurements;
   wherein the vital measurement sequence comprises a plurality of vital measurements during a preceding time period preceding a prediction period;
utilizing, by the at least one processor, a recurrent neural network to predict a probability of a low risk classification for the prediction period based at least in part on the plurality of vital measurements of the preceding time period;
producing, by the at least one processor, a risk classification for the prediction period based on a probability of the low risk classification exceeding a classification probability threshold;
   wherein the risk classification comprises one of a low-risk classification and a not-low-risk classification;
generating, by the at least one processor, a vital measurement schedule modification based on the risk classification; and
causing to display, by the at least one processor, the vital measurement schedule modification to at least one practitioner; and
   wherein the vital measurement schedule modification indicates a wake-up time associated with each patient for at least one next vital measurement.

3. A non-transitory computer readable medium having software instructions stored thereon, wherein execution of the software instructions cause at least one processor to perform steps to:
receive patient vital measurement data for each patient of a plurality of patients;
   wherein the each patient vital measurement data comprises a vital measurement time-sequence, comprising:
      i) respiration rate measurements,
      ii) heart rate measurements,
      iii) blood pressure measurements, and
      iv) temperature measurements;
   wherein the vital measurement sequence comprises a plurality of vital measurements during a preceding time period preceding a prediction period;
utilize a recurrent neural network to predict a probability of a low risk classification for the prediction period based at least in part on the plurality of vital measurements of the preceding time period;
produce a risk classification for the prediction period based on a probability of the low risk classification exceeding a classification probability threshold;
   wherein the risk classification comprises one of a low-risk classification and a not-low-risk classification;
generate a vital measurement schedule modification based on the risk classification; and
cause to display the vital measurement schedule modification to at least one practitioner; and
   wherein the vital measurement schedule modification indicates a wake-up time associated with each patient for at least one next vital measurement.

4. A system comprising:
at least one processor configured to perform steps to:
   receive patient vital measurement data for each patient of a plurality of patients;
      wherein the each patient vital measurement data comprises a vital measurement time-sequence, comprising:
         i) respiration rate measurements,
         ii) heart rate measurements,
         iii) blood pressure measurements, and
         iv) temperature measurements;
      wherein the vital measurement sequence comprises a plurality of vital measurements during a preceding time period preceding a prediction period;
   utilize a recurrent neural network to predict a probability of a low risk classification for the prediction period based at least in part on the plurality of vital measurements of the preceding time period;
   produce a risk classification for the prediction period based on a probability of the low risk classification exceeding a classification probability threshold;
      wherein the risk classification comprises one of a low-risk classification and a not-low-risk classification;
   generate a vital measurement schedule modification based on the risk classification; and
   cause to display the vital measurement schedule modification to at least one practitioner; and
      wherein the vital measurement schedule modification indicates a wake-up time associated with each patient for at least one next vital measurement.

5. The method of clauses 1-4, wherein the recurrent neural network comprises Long Short-Term Memory (LSTM) cells.

6. The method of clause 5, wherein the LSTM cells are arranged in five recurrent layers.

7. The method of clauses 1-4, wherein each risk score comprises a value between 0 and 15.

8. The method of clause 1-4, wherein the risk threshold comprises 7.

9. The method of clauses 1-4, wherein the low-risk classification represents a predicted risk score of less than 7.

10. The method of clauses 1-4, further comprises determining the vital measurement schedule modification comprising a reduced wake-up schedule that removes one or more scheduled vital measurements during an overnight period when the risk classification comprises a low-risk classification.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

1. A method comprising:
receiving, by at least one processor, patient vital measurement data for each patient of a plurality of patients;
wherein the patient vital measurement data for each patient comprises a vital measurement time-sequence of vital measurements through time;
wherein the patient vital measurement data comprises a plurality of health measurements;
determining, by the at least one processor, a sub-score for each vital measurement of the vital measurements through time for each patient based on the patient vital measurement data;
determining, by the at least one processor, a risk score for each vital measurement of the vital measurements through time for each patient based on a sum of each sub-score for each vital measurement to produce a record of risk scores through time for each patient;
receiving, by the at least one processor, a label for each predetermined time period of the vital measurement time-sequence based on a comparison of each risk score in each predetermined time period with a risk threshold;
wherein the label comprises a low risk label where one or more risk scores of each vital measurement is below the risk threshold;
utilizing, by the at least one processor, a sleeping pattern modelling recurrent neural network to model a probability of a low risk classification for each predetermined time period based at least in part on a subset of the vital measurements preceding each predetermined time period;
producing, by the at least one processor, a risk classification for each predetermined time period based on a respective probability of the low risk classification exceeding a classification probability threshold;
wherein the risk classification comprises a low-risk classification, a not-low-risk classification, or a combination thereof;
determining, by the at least one processor, classification error based on a comparison of a respective risk classification and a respective label for each predetermined time period;
wherein an incorrect low-risk classification is weighted eight times more than an incorrect not-low-risk classification; and
determining, by the at least one processor, updated weights of long short-term memory (LSTM) cell of the sleeping pattern modelling recurrent neural network based on the classification error to product a trained sleeping pattern modelling recurrent neural network;
utilizing, by the at least one processor, the trained sleeping pattern recurrent neural network to produce, for each patient, a patient-specific vital measurement schedule modification based on the patient vital measurement data;
causing to display, by the at least one processor, the patient-specific vital measurement schedule modification for each patient to at least one practitioner; and
wherein each patient-specific vital measurement schedule modification indicates at least one action related to sleeping patterns.

2. The method of claim 1, wherein the recurrent neural network comprises Long Short-Term Memory (LSTM) cells.

3. The method of claim 2, wherein the LSTM cells are arranged in five recurrent layers.

4. The method of claim 1, wherein each risk score comprises a value between 0 and 15.

5. The method of claim 1, wherein the risk threshold comprises 7.

6. The method of claim 1, wherein the low-risk classification represents a predicted risk score of less than 7.

7. The method of claim 1, further comprises determining the vital measurement schedule modification comprising a reduced wake-up schedule that removes one or more scheduled vital measurements during an overnight period when the risk classification comprises a low-risk classification.

8. A method comprising:
receiving, by at least one processor, patient vital measurement data for each patient of a plurality of patients;
wherein the patient vital measurement data comprises a vital measurement time-sequence of vital measurement through time;
wherein the patient vital measurement data comprises a plurality of health measurements;
wherein the vital measurement time-sequence comprises a plurality of the vital measurements during a preceding time period preceding a prediction period;
utilizing, by the at least one processor, a trained sleeping pattern recurrent neural network to predict a probability of a low risk classification for the prediction period based at least in part on the plurality of the vital measurements during the preceding time period;
producing, by the at least one processor, a risk classification for the prediction period based on a probability of the low risk classification exceeding a classification probability threshold;
wherein the risk classification comprises a low-risk classification or a not-low-risk classification, or a combination thereof;
generating, by the at least one processor, a vital measurement schedule modification based on the risk classification; and
causing to display, by the at least one processor, the vital measurement schedule modification to at least one practitioner; and
wherein each patient-specific vital measurement schedule modification indicates at least one action related to sleeping patterns.

9. The method of claim 8, wherein the recurrent neural network comprises Long Short-Term Memory (LSTM) cells.

10. The method of claim 9, wherein the LSTM cells are arranged in five recurrent layers.

11. The method of claim 8, wherein each risk score comprises a value between 0 and 15.

12. The method of claim 8, wherein the risk threshold comprises 7.

13. The method of claim 8, wherein the low-risk classification represents a predicted risk score of less than 7.

14. The method of claim 8, further comprises determining the vital measurement schedule modification comprising a reduced wake-up schedule that removes one or more scheduled vital measurements during an overnight period when the risk classification comprises a low-risk classification.

15. A non-transitory computer readable medium having software instructions stored thereon, wherein execution of the software instructions cause at least one processor to perform steps to:
receive patient vital measurement data for each patient of a plurality of patients;
wherein the patient vital measurement data comprises a vital measurement time-sequence of vital measurements through time;
wherein the patient vital measurement data comprises a plurality of health measurements;
wherein the vital measurement time-sequence comprises a plurality of the vital measurements during a preceding time period preceding a prediction period;
utilize a trained sleeping pattern recurrent neural network to predict a probability of a low risk classification for the prediction period based at least in part on the plurality of the vital measurements of the preceding time period;
produce a risk classification for the prediction period based on a probability of the low risk classification exceeding a classification probability threshold;
wherein the risk classification comprises one of a low-risk classification and a not-low-risk classification;
generate a vital measurement schedule modification based on the risk classification; and
cause to display the vital measurement schedule modification to at least one practitioner; and
wherein each patient-specific vital measurement schedule modification indicates at least one action related to sleeping patterns.

16. The non-transitory computer readable medium of claim 15, wherein the recurrent neural network comprises Long Short-Term Memory (LSTM) cells.

17. The non-transitory computer readable medium of claim 16, wherein the LSTM cells are arranged in five recurrent layers.

18. The non-transitory computer readable medium of claim 15, wherein each risk score comprises a value between 0 and 15.

19. The non-transitory computer readable medium of claim 15, wherein the risk threshold comprises 7.

20. The non-transitory computer readable medium of claim 15, wherein the low-risk classification represents a predicted risk score of less than 7.

21. The non-transitory computer readable medium of claim 15, further comprises determining the vital measurement schedule modification comprising a reduced wake-up schedule that removes one or more scheduled vital measurements during an overnight period when the risk classification comprises a low-risk classification.

22. A system comprising:
at least one processor configured to perform steps to:
receive patient vital measurement data for each patient of a plurality of patients;
wherein the patient vital measurement data comprises a vital measurement time-sequence of vital measurements through time;
wherein the patient vital measurement data comprises a plurality of health measurements;
wherein the vital measurement sequence comprises a plurality of the vital measurements during a preceding time period preceding a prediction period;
utilize a trained sleeping pattern recurrent neural network to predict a probability of a low risk classification for the prediction period based at least in part on the plurality of vital measurements of the preceding time period;
produce a risk classification for the prediction period based on a probability of the low risk classification exceeding a classification probability threshold;
wherein the risk classification comprises one of a low-risk classification and a not-low-risk classification;
generate a vital measurement schedule modification based on the risk classification; and
cause to display the vital measurement schedule modification to at least one practitioner; and
wherein each patient-specific vital measurement schedule modification indicates at least one action related to sleeping patterns.

23. The system of claim 22, wherein the recurrent neural network comprises Long Short-Term Memory (LSTM) cells.

24. The system of claim 23, wherein the LSTM cells are arranged in five recurrent layers.

25. The system of claim 22, wherein each risk score comprises a value between 0 and 15.

26. The system of claim 22, wherein the risk threshold comprises 7.

27. The system of claim 22, wherein the low-risk classification represents a predicted risk score of less than 7.

28. The system of claim 22, wherein the at least one processor is further configured to perform steps to determine the vital measurement schedule modification comprising a reduced wake-up schedule that removes one or more scheduled vital measurements during an overnight period when the risk classification comprises a low-risk classification.
